(19) United States
(12) Patent Application Publication
(10) Pub. No.: US 2024/0189287 A1
(43) Pub. Date: Jun. 13, 2024

(54) PERIPHERALLY RESTRICTED CB1 RECEPTOR BLOCKERS AND USES THEREOF

(71) Applicant: BIONANOSIM (BNS) LTD, Jerusalem (IL)

(72) Inventors: Simon BENITA, Tel Aviv (IL); Joseph TAM, Jerusalem (IL); Taher NASSAR, Kfar Tur'an (IL); Noam FREEMAN, Modiin (IL)

(73) Assignee: BIONANOSIM (BNS) LTD, Jerusalem (IL)

(21) Appl. No.: 18/549,193

(22) PCT Filed: Mar. 10, 2022
(86) PCT No.: PCT/IL2022/050276

§ 371 Date: Sep. 6, 2023

Related U.S. Application Data
(60) Provisional application No. 63/160,114, filed on Mar. 12, 2021.

(51) Int. Cl. A61K 31/4196; A61K 31/4162; A61K 31/4178; A61K 31/4406; A61K 31/4439; A61K 31/454; A61K 31/4545; A61K 31/497; A61K 31/506; A61P 3/04; C07D 491/044

(52) U.S. Cl. CPC A61K 31/4196; A61K 31/4162; A61K 31/4178; A61K 31/4406; A61K 31/4439; A61K 31/454; A61K 31/4545; A61K 31/497; A61K 31/506; A61P 3/04; C07D 491/044

(57) ABSTRACT

Provided is a class of compounds generally referred to as peripherally restricted CB₁ receptor blockers. Further provided are the structural and functional properties of these compounds, and their specific applications to various clinical disorders and subclinical conditions.

### TECHNOLOGICAL FIELD

The invention generally concerns novel peripherally restricted CB₁ receptor blockers and uses thereof.

### BACKGROUND OF THE INVENTION

Obesity is a chronic disease reaching epidemic proportions, with more than one-third (34.9% or 78.6 million) of U.S. adults being diagnosed as obese. Obesity has been described as a catalyst for a number of clinical conditions, most notably cardiovascular disease, type 2 diabetes mellitus (T2DM) and non-alcoholic fatty liver disease (NAFLD). While several metabolic factors have been linked to the development of obesity, their underlying molecular mechanisms are not yet fully understood.

Endocannabinoids (eCBs) are endogenous lipid ligands interacting with the CB₁ and CB₂ cannabinoid receptors which are also receptors for 49-tetrahydrocannabinol (THC), the psychoactive component of cannabis, and mediators of its biological activity. The eCBs, through activation of CB₁ receptors, have been related to a series of effects revealed in an increased appetite (the “munchies”), lipogenesis in adipose tissue and liver, insulin resistance and dyslipidemia. These effects have led to the notion that an overactive eCB/CB₁ receptor system contributes to the development of visceral obesity, T2DM and their related complications. This in turn has prompted the pharmaceutical companies to target the CB₁ receptors with newly developed blocking drugs as potential treatments for obesity, T2DM and NAFLD. The first generation of this type of compounds, rimonabant (globally acting CB₁ receptor antagonist), was proved to be effective not only in reducing body weight in obese and overweight individuals but also in ameliorating the associated metabolic abnormalities, including fatty liver, insulin resistance and T2DM [1-6]. However, due to its psychotropic side effects, such as depression, anxiety, and suicidal behavior, rimonabant was withdrawn from the market worldwide, which in turn has lessened the importance of CB₁ receptors as candidate therapeutic targets for obesity, T2DM or NAFLD.

### Cited Publications

[1] Van Gaal, L. F., Rissanen, A. M., Scheen, A. J., Ziegler, O. & Rossner, S. Effects of the cannabinoid-1 receptor blocker rimonabant on weight reduction and cardiovascular risk factors in overweight patients: 1-year experience from the RIO-Europe study. Lancet 365, 1389-1397 (2005).
[2] Pi-Sunyer, F. X., Aronne, L. J., Heshmati, H. M., Devin, J. & Rosenstock, J. Effect of rimonabant, a cannabinoid-1 receptor blocker, on weight and cardiometabolic risk factors in overweight or obese patients: RIO-North America: a randomized controlled trial. JAMA 295, 761-775 (2006).
[3] Despres, J. P., Golay, A., Sjostrom, L. & Rimonabant in Obesity-Lipids Study, G. Effects of rimonabant on metabolic risk factors in overweight patients with dyslipidemia. N Engl J Med 353, 2121-2134 (2005).
[4] Wierzbicki, A. S., et al. Rimonabant improves cholesterol, insulin resistance and markers of non-alcoholic fatty liver in morbidly obese patients: a retrospective cohort study. Int J Clin Pract 65, 713-715 (2011).
[5] Hollander, P. Endocannabinoid blockade for improving glycemic control and lipids in patients with type 2 diabetes mellitus. The American journal of medicine 120, S18-28; discussion S29-32 (2007).
[6] Randall, M. D., Kendall, D. A., Bennett, A. J. & O'Sullivan, S. E. Rimonabant in obese patients with type 2 diabetes. Lancet 369, 555 (2007).
[7] US Patent Publication No. 2003/0199536.

### General Description

The presently disclosed technology revives the earlier prospect of CB₁ receptor blockade as a therapeutic approach to obesity, and metabolic syndromes at large. To retain the therapeutic benefits of the globally acting CB₁ receptor blockers on adipose tissues, T2DM or NAFLD and to avoid their CNS-mediated side effects, the inventors of the present technology have adopted a different approach, mimicking the effect of the peripherally restricted CB₁ receptor antagonists. To that end, the inventors have designed a new class of compounds that block the CB₁ receptor only in the peripheral organs, such as adipose tissue, the liver, skeletal muscles, pancreatic B-cells and kidneys, but do not penetrate the blood-brain-barrier, and thereby avoiding the CNS-mediated side effects characteristic of the globally acting CB₁ receptor blockers.

More specifically, the inventors have demonstrated a number of key features of the presently disclosed compositions, most notably: they are lipophilic compounds that bind to a CB₁ receptor; they are P-gp substrates; and/or have a brain/plasma ratio below 0.3; and/or have a diphenyl ethylene or diphenyl methylene moiety; and thus, exhibit therapeutic benefits without causing CNS-mediated side effects. Moreover, this novel class of compounds impacted on several clinical features of the metabolic syndrome.

Thus, in numerous embodiments the compounds of the invention can be articulated in terms of lipophilic derivatives of cannabinoids having a calculated LogP (partition coefficient between n-octanol and water) value ranging from 3 and 17.

Alternatively, the compounds of the invention can be articulated in terms of a group of CB₁ receptor-binding lipophilic compounds with one or more of the following characteristics:
a compound which is a P-glycoprotein (P-gp) substrate; and/or
a compound having a brain/plasma ratio below 0.3; and/or
a compound of formula (I) and its derivatives, as detailed below:

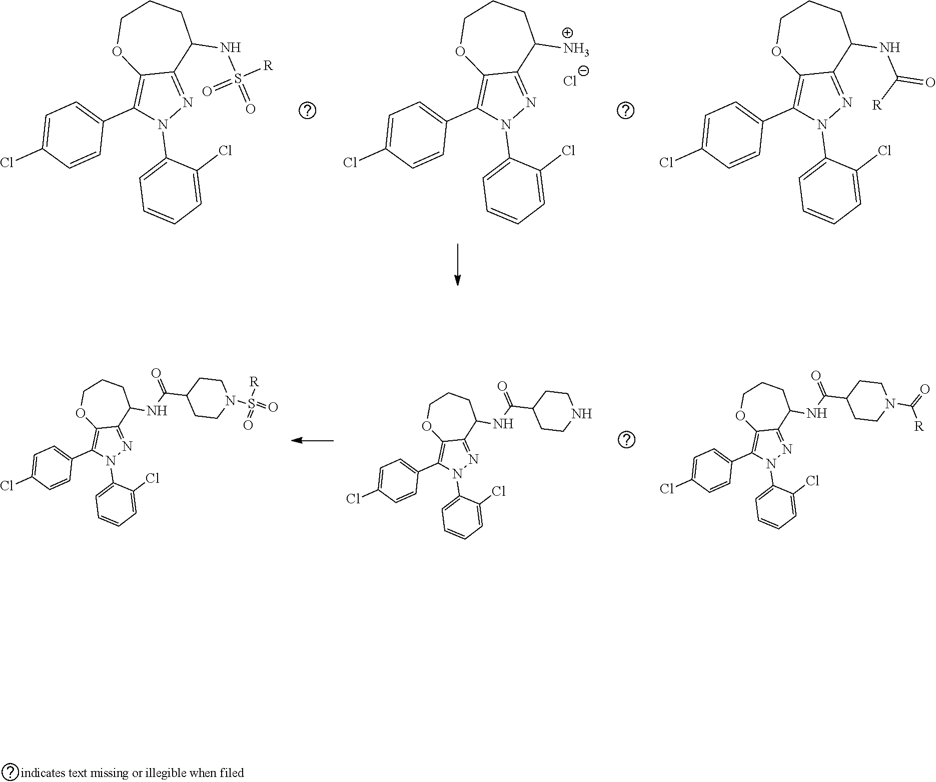

In the above, each of variables R is as defined herein.

More precisely, in some cases the CB₁ receptor-binding lipophilic compounds of the invention are P-gp substrates.

In some cases, the CB₁ receptor-binding lipophilic compounds of the invention have a brain/plasma ratio below 0.3.

In some cases, the CB₁ receptor-binding lipophilic compounds of the invention are compounds of formula (I), as disclosed.

Therapeutic benefits of the compounds of the invention stem from their ability to retain CB₁ receptor-binding without causing CNS-mediated side effects. This is because of, inter alia, their action as P-gp substrates or their interaction with P-gp which limits or significantly reduces their penetration to the brain. The absence of or a reduced penetration to the brain may be qualitatively and/or quantitatively determined by conventional methods known in the art.

For example, one of the available tools for estimation of CNS pharmacokinetics is the brain-plasma concentration ratio, which is indicative of the blood-brain barrier availability of compounds in reflecting the free drug concentration of a compound in the brain that causes the relevant pharmacological response at the target site. As was noted, compounds of the invention exhibit substantially no brain penetration.

Lipophilicity of the compounds of the invention is reflected in a calculated LogP (partition coefficient between n-octanol and water) value of these compounds, ranging from 3 and 17.

Flexibility or adaptability of the compounds of the invention to future modifications stems from their general structure of formula (I):

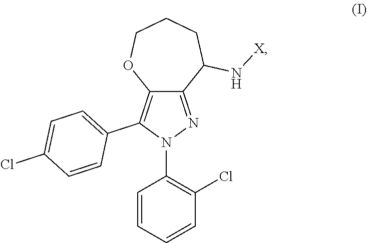

wherein
X is a group selected from —SO₂—R₁, —(C═O)—R₁, —(C═O)-Cyc-(C═O)—R₁, —(C═O)-Cyc-R₁, —(C═O)-Cyc-SO₂—R₁, and —(C═O)-Cyc.

In some embodiments, each R₁ is selected independently from —C₁-C₅alkyl, —C₆-C₁₀aryl, —C₃-C₆ heteroaryl, —C₅-C₁₀carbocyclic, —C₃-C₆heterocarbocyclyl and NRR′R″, wherein each of R, R′ and R″ independently is selected from —C₁-C₅ alkyl, —C₆-C₁₀aryl, —C₃-C₆heteroaryl, —C₅-C₁₀carbocyclic, —C₃-C₆heterocarbocyclyl, —C₆-C₁₀arylene, —C₃-C₆ heteroarylene, which may be substituted or unsubstituted.

As used herein, “Cyc” designates any cyclic moiety which may be an end of chain cyclic group or a mid-chain cyclic group, and which may be substituted or unsubstituted. The cyclic moiety may be or my comprise a group selected from —C₃-C₆carbocyclyls (cycloalkyls and cycloalkylenes), —C₃-C₆heterocarbocyclyls, —C₆-C₁₀aryls and arylenes, —C₃-C₆heteroaryls and heteroarylenes, or any substituted or unsubstituted cyclic moiety.

Exemplary cyclic moieties may include cyclohexyl, cyclopentyl, phenyl, azeridinyl, oxiranyl, pyrrolidinyl, pyrrolyl, furanyl, thiophenyl, piperidinyl, oxanyl, pyridinyl, pyranyl, imidazonlidinyl, pyrazolinyl, imidazolinyl, pyrazolyl, imidazolyl, triazolyl, isozazolyl, tetrahydropyranyl, pyranyl, morpholinyl, oxazinyl, and others.

In some embodiments, R₁ is selected amongst cyclic-containing moieties.

In some embodiments, R₁ is selected amongst acyclic moieties.

In some embodiments, the cyclic moiety is a mid-chain moiety, namely in a form of a cyclylene. In some embodiments, the cyclic moiety is an end of chain moiety in a form of a cyclyl.

In some embodiments, each of group R₁ is substituted by one or more functional groups. In some embodiments, the substituting functional groups are selected from halides (Cl, Br, I and/or F), hydroxyl groups, amines, nitro groups, nitrile groups, sulfoxide groups, sulfonyl groups, alkyl groups, alkenyl groups, alkynyl groups, trifluorinated groups, aldehyde groups, ester groups, ketone groups, amide groups, oxygen radical groups and others.

As used herein, the moiety “—C₁-C₅alkyl” refers to a carbon chains containing from 1 to 5 carbon atoms, inclusive, that are straight or branched. Exemplary alkyl groups include, but are not limited to, methyl, ethyl, propyl, isopropyl, isobutyl, n-butyl, sec-butyl, tert-butyl, and pentyl groups.

The moiety “—C₆-C₁₀aryl” or “—C₆-C₁₀arylene” refers to aromatic monocyclic or multicyclic groups containing from 6 to 10 carbon atoms. Aryl groups include but are not limited to groups such as unsubstituted or substituted phenyl, benzyl, fluorenyl, naphthyl and substituted forms of each.

The moiety “—C₃-C₆heteroaryl” or “—C₃-C₆heteroarylene” refers to a monocyclic or multicyclic aromatic ring system having between 3 and 10 atoms, wherein one or more, in some embodiments 1 to 3, of the atoms in the ring system is a heteroatom selected from nitrogen, oxygen and sulfur. The heteroaryl group may be optionally fused to a benzene ring. Heteroaryl groups include, but are not limited to, furyl, imidazolyl, pyrimidinyl, tetrazolyl, thienyl, pyridyl, pyrrolyl, thiazolyl, isothiazolyl, oxazolyl, isoxazolyl, triazolyl, quinolinyl and isoquinolinyl,

The moiety “—C₅-C₁₀carbocyclic” or “cycloalkyl” refers to a saturated mono- or multi-cyclic ring system of 5 to 10 carbon atoms. The ring systems may be composed of one ring or two or more rings which may be joined together in a fused, bridged or spiro-connected fashion.

The moiety “—C₃-C₆heterocarbocyclyl” or “heterocyclyl” refers to a monocyclic or multicyclic non-aromatic ring system, containing between 3 and 10 atoms, wherein one or more, in some embodiments, 1 to 3, of the atoms in the ring system is a heteroatom, selected from nitrogen, oxygen and sulfur. In embodiments where the heteroatom(s) is(are) nitrogen, the nitrogen is optionally substituted with alkyl, alkenyl, alkynyl, aryl, heteroaryl, aralkyl, heteroaralkyl, cycloalkyl, heterocyclyl, cycloalkylalkyl, heterocyclylalkyl, acyl, guanidine, or the nitrogen may be quaternized to form an ammonium group where the substituents are selected as above.

The moiety —NRR′R″ refers to a nitrogen containing groups such as an amine, wherein each of R, R′ and R″, independently of the other, is selected as above. The amine may be a primary, secondary or a tertiary amine.

It is to be understood that the compounds provided herein may contain chiral centers. Such chiral centers may be of either the (R) or (S) configuration or may be a mixture thereof. Thus, the compounds provided herein may be enantiomerically pure, or be stereoisomeric or diastereomeric mixtures. It is to be understood that the chiral centers of the compounds provided herein may undergo epimerization in vivo. As such, one of skill in the art will recognize that administration of a compound in its (R) form is equivalent, for compounds that undergo epimerization in vivo, to administration of the compound in its (S) form.

Compounds of the invention may be presented as salts, i.e., as pharmaceutically acceptable salts.

Acid addition salts of compounds of the invention include salts derived from inorganic acids such as hydrochloric, nitric, phosphoric, sulfuric, hydrobromic, hydriodic, phosphorous, and the like, as well as the salts derived from organic acids, such as aliphatic mono- and dicarboxylic acids, phenyl-substituted alkanoic acids, hydroxy alkanoic acids, alkanedioic acids, aromatic acids, aliphatic and aromatic sulfonic acids, etc. Such salts thus include sulfate, pyrosulfate, bisulfate, sulfite, bisulfite, nitrate, phosphate, monohydrogenphosphate, dihydrogenphosphate, metaphosphate, pyrophosphate, chloride, bromide, iodide, acetate, propionate, caprylate, isobutyrate, oxalate, malonate, succinate, suberate, sebacate, fumarate, maleate, mandelate, benzoate, chlorobenzoate, methylbenzoate, dinitrobenzoate, phthalate, benzenesulfonate, toluenesulfonate, phenylacetate, citrate, lactate, maleate, tartrate, methanesulfonate, and the like. Also contemplated are salts of amino acids such as arginate and the like and gluconate, galacruronate (see, for example, Berge S. M., ct al., “Pharmaceutical Salts,” J. of Pharmaceutical Science, 66:1-19 (1977)).

The acid addition salts of basic compounds may be prepared by contacting the free base form with a sufficient amount of the desired acid to produce the salt in the conventional manner. The free base form may be regenerated by contacting the salt form with a base and isolating the free base in the conventional manner. The free base forms differ from their respective salt forms somewhat in certain physical properties such as solubility in polar solvents, but otherwise the salts are equivalent to their respective free base for purposes of the present invention. Pharmaceutically acceptable base addition salts are formed with metals or amines, such as alkali and alkaline earth metals or organic amines. Examples of metals used as cations are sodium, potassium, magnesium, calcium, and the like. Examples of suitable amines are N,N′-dibenzylethylenediamine, chloroprocaine, choline, diethanolamine, ethylenediamine, N-methylglucamine, and procaine (see, for example, Berge S. M., et al., “Pharmaceutical Salts,” J. of Pharmaceutical Science, 66:1-19 (1977)).

The base addition salts of acidic compounds are prepared by contacting the free acid form with a sufficient amount of the desired base to produce the salt in the conventional manner. The free acid form may be regenerated by contacting the salt form with an acid and isolating the free acid in the conventional manner. The free acid forms differ from their respective salt forms somewhat in certain physical properties such as solubility in polar solvents, but otherwise the salts are equivalent to their respective free acid for purposes of the present invention.

Wherever a salt form or a stereospecific form of a compound of the invention is depicted herein, the free acid or free base or stereo unspecified forms are also included. For example, a compound of the structure (I) wherein R₁ is the moiety

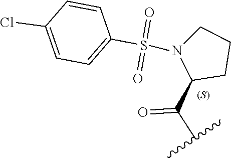

also encompasses a compound of structure (I), wherein the chiral center is (R) and wherein the chiral center is unspecified. Similarly, a compound of the structure (I), wherein R₁ is

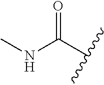 [SMILES: *C(=O)NC]

also encompasses a salt form thereof (wherein the N atom is protonated).

Compounds of the invention thus include free forms thereof, salt forms thereof, stereospecific or stereo unspecific forms thereof, and hydrated forms thereof.

In some embodiments, X is selected from

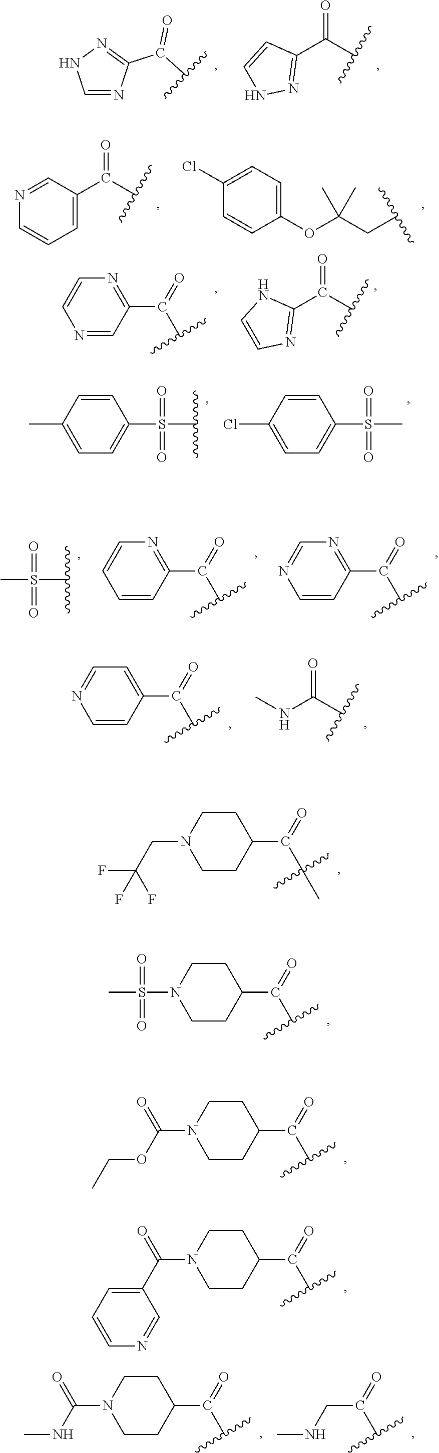

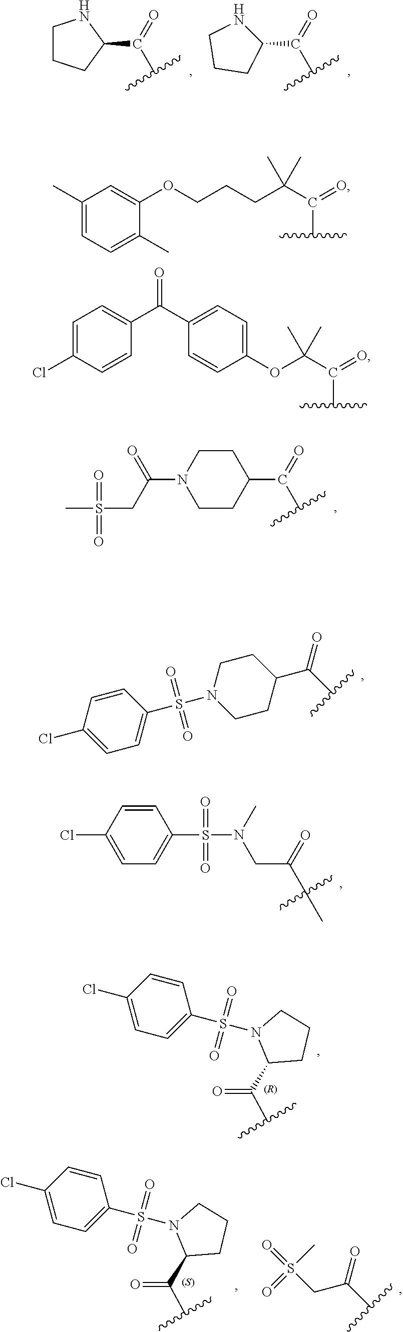

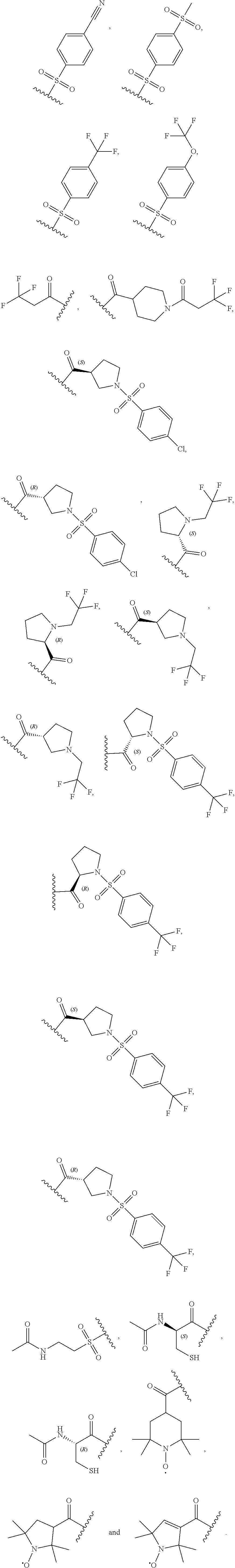

Specific non limiting examples of such compounds are compounds designated herein as BNS801 through BNS828. Compounds BNS801 through BNS828 are encompassed as free acids, free bases, stereospecific or unspecific or as salts. Compounds BNS801 through BNS828 are compound of structure (1) having X listed below:

| ID # | X |
|---|---|
| BNS801 | 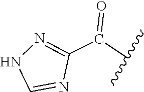 [SMILES: *C(=O)c1nc[nH]n1] |
| BNS802 | 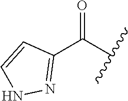 [SMILES: *C(=O)c1cc[nH]n1] |
| BNS803 | 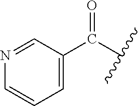 [SMILES: *C(=O)c1cccnc1] |
| BNS804 | 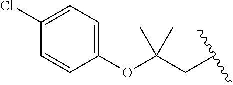 [SMILES: *CC(C)(C)Oc1ccc(Cl)cc1] |
| BNS805 | 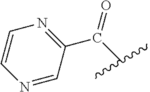 [SMILES: *C(=O)c1cnccn1] |
| BNS806 | 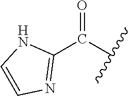 [SMILES: *C(=O)c1ncc[nH]1] |
| BNS807 | 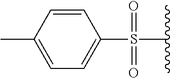 [SMILES: *S(=O)(=O)c1ccc(C)cc1] |
| BNS808 | 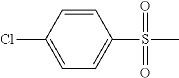 |
| BNS809 | 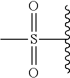 [SMILES: *S(C)(=O)=O] |
| BNS810 | 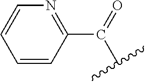 [SMILES: *C(=O)c1ccccn1] |
| BNS811 | 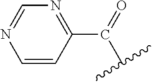 [SMILES: *C(=O)c1ccncn1] |
| BNS812 | 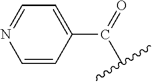 [SMILES: *C(=O)c1ccncc1] |
| BNS813 | 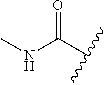 [SMILES: *C(=O)NC] |
| BNS814 | 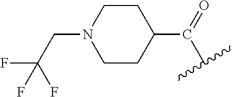 [SMILES: *C(=O)C1CCN(CC(F)(F)F)CC1] |
| BNS815 | 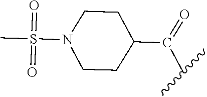 [SMILES: *C(=O)C1CCN(S(C)(=O)=O)CC1] |
| BNS816 | 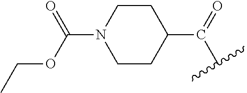 [SMILES: *C(=O)C1CCN(C(=O)OCC)CC1] |
| BNS817 | 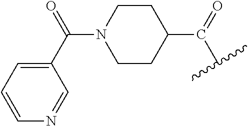 [SMILES: *C(=O)C1CCN(C(=O)c2cccnc2)CC1] |
| BNS818 | 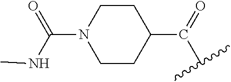 [SMILES: *C(=O)C1CCN(C(=O)NC)CC1] |
| BNS819 | 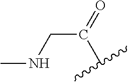 [SMILES: *C(=O)CNC] |
| BNS820 | 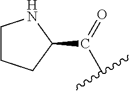 [SMILES: *C(=O)[C@H]1CCCN1] |
| BNS821 | 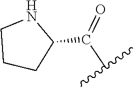 [SMILES: *C(=O)[C@@H]1CCCN1] |
| BNS822 | 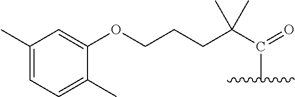 [SMILES: *C(=O)C(C)(C)CCCOc1cc(C)ccc1C] |
| BNS823 | 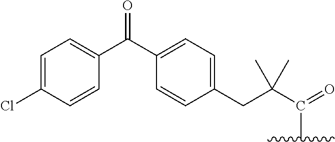 [SMILES: *C(=O)C(C)(C)Cc1ccc(C(=O)c2ccc(Cl)cc2)cc1] |
| BNS824 | 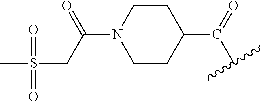 [SMILES: *C(=O)C1CCN(C(=O)CS(C)(=O)=O)CC1] |
| BNS825 | 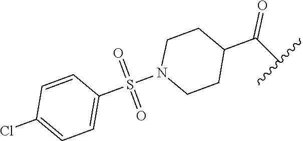 [SMILES: *C(=O)C1CCN(S(=O)(=O)c2ccc(Cl)cc2)CC1] |
| BNS826 | 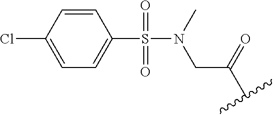 [SMILES: *C(=O)CN(C)S(=O)(=O)c1ccc(Cl)cc1] |
| BNS827 | 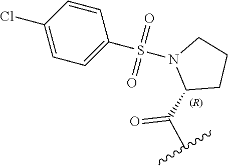 |
| BNS828 | 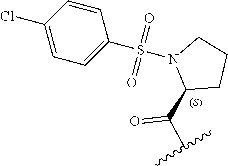 |

Each of the above listed compounds constitutes a separate embodiment of the invention.

In some embodiments, the compound is a compound herein designated BNS802 and compound BNS822.

In some embodiments, a compound of the invention is a compound of formula (I), excluding compounds BNS802 through BNS813 and BNS817, BNS818, BNS820 and BNS822.

The invention further provides uses of compounds of structure (I) and compositions comprising same, as disclosed herein. The uses and compositions disclosed herein include a compound as disclosed herein, being in some embodiments, a compound designated herein as BNS801 through BNS828, each constituting a separate embodiment of the invention.

Compounds of the invention can be described as modulators of peripheral cannabinoid receptors, including peripherally restricted CB₁ receptors and CB₂ receptors. In some embodiments, the compounds can be modulators, and in many cases inhibitors, of a peripherally restricted CB₁ receptor. In some cases, the compounds can be neutral antagonists or inverse agonists. In some cases, the compounds can be modulators or activators of CB₂ receptors.

Peripherally restricted CB₁ receptor blockers generally refer to agents/materials that are antagonists or blockers of CB₁ receptors present in peripheral organs and tissues, including the adipose tissues, the liver, skeletal muscles, pancreatic β-cells and the kidneys, without causing CNS-mediated side effects. In the context of the invention, these blockers or antagonists can retain the therapeutic benefits of globally acting CB₁ receptor blockers without causing CNS-mediated side effect. Specific non limiting examples of such compounds are compounds designated herein as BNS801 through BNS828.

From a clinical point of view, a CB₁ receptor blocker or antagonist, either a partial or a full blocker, by virtue of inhibiting or neutralizing the biological function of a peripheral CB₁ receptor, can be used in the prevention or treatment of various metabolic syndromes, from obesity, insulin resistance, diabetes to coronary heart disease, fatty liver, hepatic cirrhosis, chronic kidney disease and cancer.

The presently disclosed compounds can serve as a basis for a series of products generally referred to as pharmaceutical compositions that can be further adapted for various modes of administration for various clinical applications in humans and animals. Generally, pharmaceutical compositions comprise a therapeutically effective amount of an active, i.e., a compound of the invention, together with one or more pharmaceutically acceptable additive such as diluents, buffers, preservatives, solubilizers, emulsifiers, adjuvant and/or carriers. The pharmaceutical compositions may be formulated as liquids or lyophilized or otherwise dried formulations.

Pharmaceutical compositions can be further adapted for various modes of administration. Oral compositions can be introduced in a variety of forms, such as (a) liquid solutions, (b) capsules, sachets, tablets, lozenges, and troches, (c) powders, (d) suspensions, and (e) emulsions or self-emulsifying formulations, using known in the art additives and formulation technologies.

Pharmaceutical compositions for parenteral administration can include sterile nanoemulsions, aqueous and non-aqueous, isotonic sterile injection solutions with antioxidants, buffers, bacteriostats, and isotonic solutes, and aqueous and non-aqueous sterile suspensions with solubilizers, thickening agents, stabilizers, and preservatives. The requirements for effective formulation and specific carriers compatible with injectable pharmaceutical compositions are well known in the art.

In other words, using various know in the art technologies, the presently disclosed compounds can be used for the design of oral and injectable formulations, the latter can be further adapted for administering via intravenous or intramuscular, subcutaneous, intraperitoneal routes.

Of particular interest for oral and injectable applications are self-emulsifying oil formulations of nanocarriers (usually up to 700 nm) with the compounds to the invention. A nanocarrier usually implies a biocompatible particulate material that is sufficiently resistant to chemical and/or physical destruction, or in other words, after administration into the body, the nanocarriers material remains substantially intact for a sufficient time, such that a sufficient amount of the nanocarriers material is able to reach the target tissue or organ. The nanocarriers can be nanoparticles, nanocapsules or a mixture of both.

For chosen applications and depending on specific requirements of solubility, molecular weight, polarity, electrical charge, reactivity, chemical stability, biological activity, and others, this type formulations can be further encapsulated into nanocapsules (NCs) and/or embedded into a core/shell structure or a matrix of nanoparticles (NPs) or in regular or nano self-emulsifying delivery systems.

In other words, the nanocarriers with the compounds of the invention can be comprised in a core/shell structure (i.e., nanocapsules) which in itself is a nanoparticle or in a nanoparticle without a distinct core/shell structure (nanoparticles, NPs). The nanocarriers can be further encapsulated within a second shell layer or matrix comprised of the same or different material for a double-layered protection. Encapsulation techniques and specific requirements of materials for forming nanocarriers, nanocapsules and NPs are well known in the art. Examples are polyesters including polylactic acid (PLA), polyglycolic acid (PGA), and copolymers of (PLA/PGA).

One important advantage of formulation via nanonization and encapsulation is the ability to pack a plurality of nanocarriers in a single encasing and thereby to increase drug loading and the amount of active reaching the target tissue or organ, in other words, increasing drug efficacy overall.

Another important feature of nanonized or encapsulated formulations is the ability to exhibit long-acting or sustained/controlled actives release profiles, which can be further enhanced by incorporation of specific materials in the core/shell or matrix of NPs.

Yet another feature specific nanonized or micronized formulations, and specifically powder formulations, is their compatibility with delivery via inhalation, including oral, mucosal and/or pulmonary delivery. One example of such inhalation compatible preparations is formulations of nanocarriers comprised in NPs made of a hydrophobic polymer.

Ultimately, the compounds of the invention form the basis for a series of methods, uses and clinical applications for primary, secondary and tertiary therapeutic prevention or treatment of diseases and disorders associated with CB₁ receptor activity, such as disorders from the group of metabolic diseases, cardiovascular disease and conditions, cancer and others. Notable examples of metabolic diseases or syndromes are obesity, insulin resistance, diabetes, coronary heart disease, fatty liver disease, chronic kidney disease and liver cirrhosis.

More generally, the methods of the invention can serve the purpose of reducing body fat or body weight, reducing or controlling high blood pressure, improving a poor lipid profile, i.e., elevated LDL cholesterol/low HDL cholesterol/elevated triglycerides.

Overall, the presently disclosed compounds, sharing the properties of high lipophilicity, blocking the CB₁ receptors and little brain penetration, hold out the prospect of capturing many if not all the therapeutic properties of the globally acting CB₁ receptor blockers without the CNS-mediated side effects. The present experimental data using clinical and behavioral paradigms provide multiple lines of evidence that these compounds may constitute promising new therapies for controlling obesity and related metabolic function/liver disease and other disorders.

### BRIEF DESCRIPTION OF THE DRAWINGS

To better understand the subject matter and to exemplify how it may be carried out, embodiments are now described by way of examples, which are not meant be limiting, with reference to the following drawings.

FIGS. 1A-1C illustrate the effect of continued PO administration of the compounds of the invention on the body weight of DIO (Diet-Induced-Obese) mice, showing a significant decrease in the total body weight (%) (1A) and corresponding decrease in fat mass (1B) and increase in lean mass (1C) of mice BNS808-treated mice (1 mg/kg/day) (grey) compared to vehicle-treated control (Veh) (black). Data represents mean±SEM (N=4-6 per group).

FIGS. 2A-2F illustrate the effect of continued PO administration of the compounds on metabolic parameters of DIO mice, measuring respiratory quotient (2A), VO₂ (2B), VCO₂ (2C), total energy expenditure (TEE) (2D), fat oxidation (2E) and carbohydrate oxidation (2F) over 24 hr in BNS808-treated mice (1 mg/kg/day) (grey) and Veh-treated control (black). Data represents mean±SEM (N=4-6 per group and *P<0.05 vs. Veh).

FIGS. 3A-3D illustrate the effect of continued PO administration of the compounds on ambulation parameters of DIO mice, measuring ambulatory activity (3A), ability to run on a wheel (3B), voluntary activity (3C) and total meter (3D) over 24 hr in BNS808-treated mice (1 mg/kg/day) (grey) and Veh-treated control (black), including diurnal (light) and nocturnal (dark) behaviors. Data represents mean±SEM (N=4-6 per group and *P<0.05 vs. Veh).

FIGS. 4A-4H illustrate the effect of continued PO administration of the compounds on parameters of diabetes in DIO mice, measuring glucose tolerance (GTT) (4A and 4B as Area Under Curve (AUC), fasting (4C) and fed glucose levels (4D) and insulin sensitivity (ITT, Insulin Tolerance Test) (4E and 4H as AUC) in BNS808-treated mice (1 mg/kg/day) (grey) and Veh-treated control (black). Data represents mean±SEM (N=4-6 per group).

FIGS. 5A-5E illustrate the effect of continued PO administration of the compounds on HFD-induced (High-Fat Diet) hepatic steatosis and liver injury in DIO mice, evident by H&E staining of liver sections showing a reduced deposition of fat vacuoles in the treated animals (5A) and measurements of liver triglyceride (TG) levels (5B), liver cholesterol levels (5C), and serum levels of AST and ALT (aspartate transaminase and alanine aminotransferase liver enzymes) (5D and 5E respectively) in BNS808-treated mice (1 mg/kg/day) (grey) and Veh-treated control (black). Data represents mean±SEM (N=4-6 per group and *P<0.05 vs. Veh).

FIGS. 6A-6E illustrate the effect of continued PO administration of the compounds on dyslipidemia in DIO mice, measuring serum levels of triglycerides (TG) (6A), cholesterol (6B), low-density lipoprotein (LDL) (6C), low-density lipoprotein (HDL) (6D) and HDL-to-LDL ratio (6E) in BNS808-treated mice (1 mg/kg/day) (grey) and Veh-treated control (black). Data represents mean±SEM (N=4-6 per group and *P<0.05 vs. Veh).

FIGS. 7A-7E illustrate the effect of continued PO administration of the compounds on kidney function in DIO mice, measuring the levels of plasma creatinine (CRE) (7A), blood urea nitrogen (BUN) (7B), urine CRE (7C), glomerular filtration rate (GFR) (7D) and urine glucose (7E) in mice treated with BNS808 (1 mg/kg/day) (grey) and Veh control (black). Data represents mean±SEM (N=4-6 per group and *P<0.05 vs. Veh).

FIGS. 8A-8D illustrate that an increased exposure to the compounds can lead to significant changes in the body weight of DIO mice, manifested in a reduction in body weight (8A), total food intake (8B) and fat mass (8C) and a corresponding increase in lean mass (8D) in BNS808-treated mice (20 mg/kg/day PO for 40 days) (grey) compared to Veh-treated control (white). Data represents mean±SEM (N=8 per group and *P<0.05 vs. Veh).

FIGS. 9A-9G illustrate the effect of the compounds on glycemic control in HFD mice treated with BNS822 (20 mg/kg/day PO for 20 weeks) or Veh, measuring GTT (9A and 9B as AUC), glucose levels in fed (9C) and fasting (9D), ITT (9E and 9F as AUC), and insulin plasma levels (9G). While no significant changes were observed in the glucose levels in fed and fasting and in ITT, significant reductions were observed in GTT and plasma insulin comparing treated (grey) and untreated (white) mice. Data represents mean±SEM (N=8 per group and *P<0.05 vs. Veh).

FIGS. 10A-10E illustrate the effect of the compounds on HFD-induced hepatic steatosis and liver injury in mice treated with BNS822 (20 mg/kg/day PO for 40 days) Veh, evident by H&E staining showing elevated deposition of fat vacuoles in the liver of untreated (10A) compared to treated (10B) mice, and the measurements of liver TG (10C), cholesterol (10D) and ALT (10E). Significant reductions were observed in the liver TG and ALT (10C-10E) but not the liver cholesterol levels (10D) comparing treated (grey) and untreated (white) mice. Data represents the mean±SEM (N=8 per group and *P<0.05 vs. Veh).

FIGS. 11A-11E illustrate the inducing effect of the compounds on the centrally mediated hyperactivity in mice, measuring ambulatory activity (10A and 10B), voluntary activity (10C), wheel running (10D) and all meters (10E) in C57Bl/6J mice (wild-type, males) after a single dose administration of BNS822 (20 mg/kg PO) (dark grey), rimonabant as a positive control (10 mg/kg IP) (light grey) or Veh (black). Data represents mean±SEM (N=8 per group and *P<0.05 vs. Veh).

FIGS. 12A-12D illustrate the inhibitory effect of the compounds on the hypomotility-induced by HU210 (CB₁ receptor agonist), measuring ambulatory activity (12A and 12B), voluntary activity (12C) and all meters (12D) in C57Bl/6J mice after a single dose administration of BNS822 (20 mg/kg PO) (diagonal pattern), rimonabant (10 mg/kg IP) (criss-cross pattern) or Veh (black), and after 30 min, a single dose of HU210 (30 μg/kg IP) (grey). Data represents mean±SEM (N=8 per group and *P<0.05 vs. Veh and #P<0.05 vs. HU210).

FIGS. 13A-13C illustrate the apparent lack centrally mediated effects of the compounds, measuring WIN55,212-induced catalepsy (WIN55,212 is a cannabinoid receptor agonist) (13A) and anxiogenic behavior in the Elevated Plus Maze (13B-13C) after a single dose of BNS822 (20 mg/kg PO) (black), rimonabant (10 mg/kg PO) (grey) and Veh (white) administered 30 min prior to WIN55,212 (3 mg/kg IP). Data shows that, unlike rimonabant, BNS822 had no effect on WIN55,212-induced catalepsy and no anxiogenic effect on animal behavior. Data represents mean±SEM (N=4-20 per group and *P<0.05 vs. Veh and #P<0.05 vs. rimonabant).

FIGS. 14A-14E illustrate the dose response to the treatment of the invention by the inducing effect on the centrally mediated hyperactivity, measuring ambulatory activity (14A and 14B), voluntary activity (14C), wheel running (14D) and all meters (14E) in C57Bl/6J mice after single dose administration of BNS808 (1 and 10 mg/kg PO) (block pattern and dotted pattern), rimonabant (10 mg/kg, IP) (grey) or Veh (black). Data represents mean±SEM (N=8 per group, and *P and #P<0.05 vs. Veh-treated control).

FIGS. 15A-15D illustrate dose response to the compounds by the inhibitory effect on the HU210-induced hypomotility, measuring ambulatory activity (15A and 15B), voluntary activity (15C) and all meters (15D) and all meters (14E) in C57Bl/6J mice after single dose administration of BNS822 (1 and 10 mg/kg PO) (white and grey), rimonabant (10 mg/kg IP) (pattern) or Veh (black), and after 30 min, a single dose of HU210 (30 μg/kg IP). Data represents mean±SEM (N=8 per group and *P<0.05 vs. Veh-treated control and #P<0.05 vs. HU210).

### DETAILED DESCRIPTION OF EMBODIMENTS

The compounds of the invention can be generally described as lipophilic CB₁ receptor-binding compounds. In numerous embodiments the compounds of the invention can have a calculated LogP (partition coefficient between n-octanol and water) value ranging from about 3 and about 17, or more specifically, a calculated Log P value in a range of about 3 and about 17, about 4 and about 16, about 5 and about 15, about 6 and about 14, about 7 and about 13, about 8 and about 12, about 9 and about 11, or a calculated Log P of at least about 3, at least about 4, at least about 5, at least about 6, at least about 7, at least about 8, at least about 9, at least about 10, at least about 11, at least about 12, at least about 13, at least about 14, at least about 15, at least about 16, at least about 17.

In numerous embodiments the compounds of the compounds of the invention can belong to the group of compounds of formula (I), a defined hereinabove.

According to some embodiments, provided are compounds of structure (I):

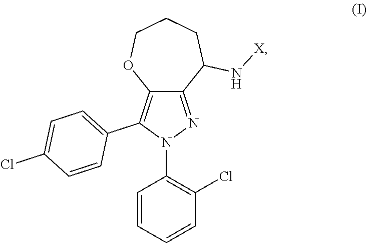

wherein
X can be a group selected from —SO₂—R₁, —(C═O)—R₁, —(C═O)-Cyc-(C═O)—R₁, —(C═O)-Cyc-R₁, —(C═O)-Cyc-SO₂—R₁, and —(C═O)-Cyc. Each of the moieties is as disclosed herein.

In some embodiments, R₁ can be selected from C₁-C₅alkyl, C₆-C₁₀ aryl, C₃-C₆ heteroaryl and NRR′R″, wherein each of R, R′ and R″ independently can be selected from C₁-C₅ alkyl, C₆-C₁₀ aryl, C₃-C₆ heteroaryl, cycloalkylene, an arylene, a heteroarylene, or any substituted or unsubstituted cyclic moiety.

In some embodiments, Cyc can be a cycloalkylene, an arylene, a heteroarylene, or any substituted or unsubstituted cyclic moiety.

In some embodiments, X can be selected from

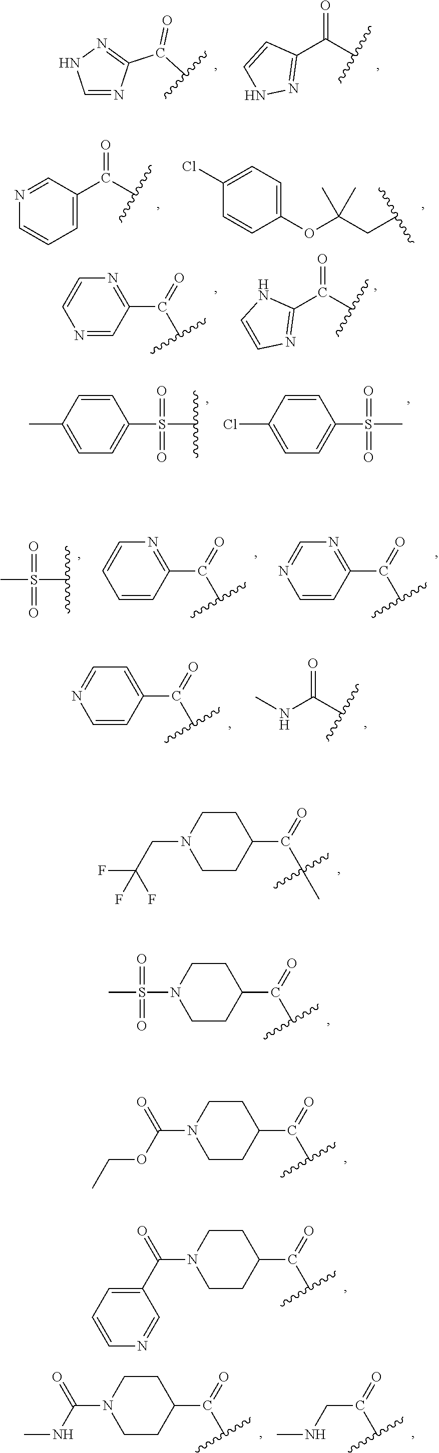

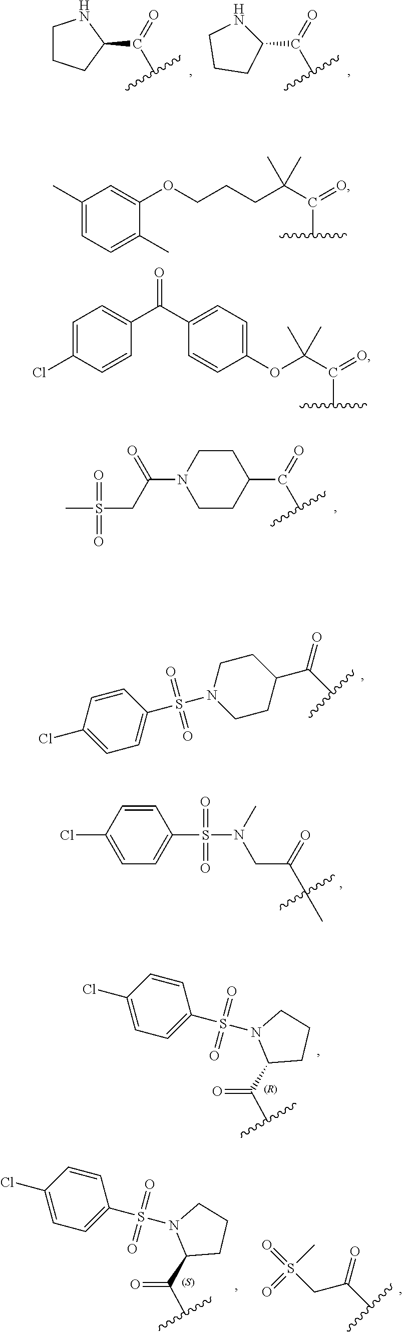

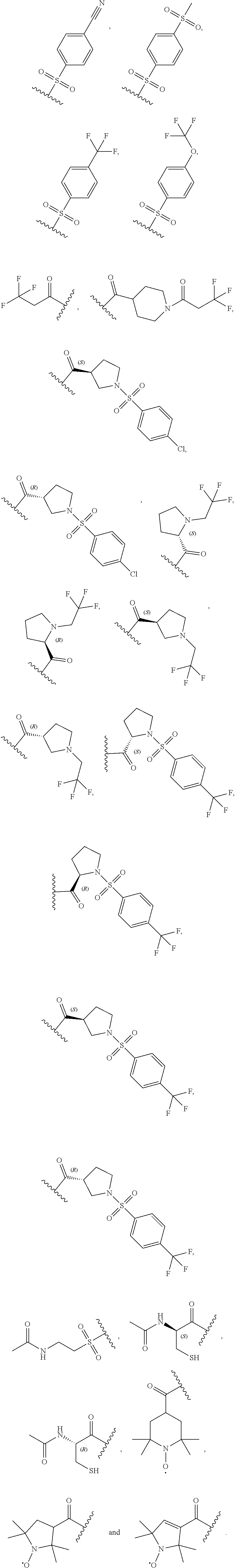

Any designation of isomers or chiral centers should be regarded as not limiting. Where a specific isomer is indicated, its stereoisomer is also included.

Methods of preparing such compounds have been presently exemplified.

In some embodiments, a compound of the invention can be a compound herein designated BNS801 through BNS828, the preparation of which has been presently exemplified.

In some embodiments, a compound of the invention is a compound of formula (I) excluding compounds BNS802 through BNS813 and BNS817, BNS818, BNS820 and BNS822.

In terms of functionality, the compounds of the invention are generally a modulators of peripheral cannabinoid receptors, including peripherally restricted CB₁ receptors and CB₂ receptors. In some embodiments, the compounds are modulators (e.g., inhibiting) of a peripherally restricted CB₁ receptor. In some embodiments, the compounds are neutral antagonists or inverse agonists. In some embodiments, the compounds are modulators (e.g., activating) of CB₂ receptors. In some embodiments, the modulator of peripheral cannabinoid receptors of the invention is a compound herein designated BNS801 through BNS828. In some embodiments, the modulator of peripheral cannabinoid receptors of the invention is a compound of formula (I), excluding compounds BNS802 through BNS813 and BNS817, BNS818, BNS820 and BNS822.

The term “peripherally restricted CB₁ receptor blocker” refers herein to the feature of the compounds in acting as antagonist or blockers of CB₁ receptors present in the peripheral organs and tissues, such as adipose tissues, the liver, skeletal muscles, pancreatic B-cells and the kidneys, without exhibiting the centrally mediated or CNS-mediated side effects. In other words, these blockers or antagonists of the invention retain the therapeutic benefits of globally acting CB₁ receptor blockers without causing CNS-mediated side effect.

In most general terms, a “CB₁ receptor blocker” or antagonist is an agent which is capable of partially or fully blocking, inhibiting or neutralizing one or more biological functions of a peripheral CB₁ receptor. By virtue of this capability, this type of agents can be applied to achieve prevention, alleviation or treatment of a variety of clinical conditions or disorders associated with the peripheral CB₁ receptor function, such as disorders and conditions belonging to the group of metabolic syndromes, notable examples of which are obesity, insulin resistance, diabetes, coronary heart disease, fatty liver, hepatic cirrhosis, chronic kidney disease and cancer, and other disorders.

In certain embodiments the compounds of the invention can be compounds of formula (I) that are peripherally restricted CB₁ receptor inverse agonists.

One of the important features of the present compositions is revealed in the preferential activity or blocking, as above, on the peripheral CB₁ receptor or CB₂ receptors, without inducing the centrally mediated side effects or effects related to the functionality of CNS such as psychotropic and neurologic effects and other examples.

In other words, the compounds of the invention have very little or almost no brain activity, mostly because they have very little or substantially no brain penetration. The term “substantially no brain penetration” is a broad term encompassing a wide range of compounds with various indices of brain permeability as reflected in brain-plasma ratio. Specifically, this term encompasses compounds with a brain-plasma ratio ranging from about 0.0001 and about 0.3, and more specifically, compounds with a brain-plasma ratio ranging from about 0.0001 and about 0.0005, about 0.0005 and about 0.001, about 0.001 and about 0.005, about 0.005 and about 0.01, about 0.01 and about 0.05, about 0.05 and about 0.1, about 0.1 and about 0.3, or compounds with a brain-plasma ratio of at least about 0.0001, at least about 0.0005, at least about 0.001, at least about 0.005, at least about 0.01, at least about 0.05, at least about 0.1, at least about 0.2, at least about 0.3, at least about 0.4, at least about 0.5.

In certain embodiments the compounds of the invention can be compounds of formula (I) as disclosed herein, with a LogP (partition coefficient between n-octanol and water) value ranging from 3 and 17 and a brain-plasma ratio ranging from about 0.0001 and about 0.3.

In certain embodiments the compounds answering these criteria can be highly lipophilic derivative of cannabinoid. The term “cannabinoid” is used herein in the most general way to encompass compounds with an affinity to the CB₁ receptor or CB₂ receptors, from natural and synthetic sources and synthetically modified natural cannabinoids (also semi-synthetic cannabinoids).

Another important feature of the present compounds is in their applicability and adaptability to formulation methods, and the prospect of using thereof in the design and development of various pharmaceutical products and drugs.

Thus, it is another objective of the invention to provide a series of compositions comprising one or more of the presently describes compounds.

In some embodiments, the compositions can be self-emulsifying oil formulations comprising the compounds of the invention.

More specifically, the compounds of the invention can be incorporated in nano or micro self-emulsifying delivery systems (SNEDDSs). Encapsulating a drug in SNEDDSs can lead to increased solubilization, stability in the gastro-intestinal (GI) tract and improved absorption, resulting in enhanced bioavailability. SNEDDSs generally consist of an oil phase, surfactant, and cosurfactant or cosolvent. Following aqueous dispersion and mild agitation, SNEDDSs spontaneously form fine oil-in-water emulsions with droplet size of 200-700 nm or below.

In other embodiments, the compositions of the invention can be self-emulsifying oil formulations comprising nanocarriers with compounds of the invention.

In certain embodiments a nanocarrier can comprise one or more of the compounds of the invention.

In further embodiments the formulations or compositions of the invention can comprise a plurality of such nanocarriers.

More specifically, the nanocarrier may be a nanoparticle, a nanocapsule or mixtures thereof. The term “nanocarrier” implies herein a particulate biocompatible material which is sufficiently resistant to chemical and/or physical destruction, such that a sufficient amount of the nanocarriers remain substantially intact for sufficient time after administration into the human or animal body, until they reach the desired target tissue or organ.

Generally, the nanocarriers have an average diameter of up to 700 nm, and specifically, an average diameter of up to about 10 nm, about 50 nm, about 100 nm, about 150 nm, about 200 nm, about 250 nm, about 300 nm, about 350 nm, about 400 nm, about 450 nm, about 500 nm, about 550 nm, about 600 nm, about 650 nm, about 700 nm, or an average diameter in the range between about 10 nm and about 100 nm, about 100 nm and about 200 nm, about 200 nm and about 300 nm, about 300 nm and about 400 nm, about 400 nm and about 500 nm, about 500 nm and about 600 nm, about 600 nm and about 700 nm.

Depending on various parameters, such as solubility, molecular weight, polarity, electrical charge, reactivity, chemical stability of the compounds, their biological activity and others, the compounds can be encapsulated or contained in nanocapsules (NCs), and/or embedded in a matrix of nanoparticles (NPs).

For the chosen applications, the nanocarriers can be in a form of core/shell (also nanocapsule) having a polymeric shell and a core containing one or more compounds of the invention.

Alternatively, the nanoparticles can be in a form of a substantially uniform composition without a distinct core/shell structure, this type of nanocarriers are referred to herein as nanoparticles (NPs).

In some embodiments, the NPs of the invention with a the plurality of embedded or encapsulated nanocarriers can be formed of a hydrophobic polymer.

Materials suitable for forming nanocarriers, nanocapsules and/or nanoparticles of the invention are polyesters including polylactic acid (PLA), polyglycolic acid (PGA), polyhydroxybutyrate and polycaprolactone), poly(orthoesters), polyanhydrides, polyamino acid, poly(alkyl cyanoacrylates), polyphophazenes, copolymers of (PLA/PGA) and asparate or polyethylene-oxide (PEO).

In some embodiments, the nanocarrier can be a nanoparticle, the nanoparticle comprising a first matrix, wherein a compound of the invention is embedded within the matrix. In other embodiments, the nanocarrier is a nanocapsule, the nanocapsule comprising a first shell encapsulating the compound of the invention or a composition comprising the compound.

The nanocarriers can be be further enveloped by another encapsulation layer, thereby forming a double-layered protection. In some embodiments, the nanocarrier can be further encapsulated within a second shell layer, which may comprise the same or different material than that of the first shell layer. In some embodiments, the nanocarrier can be further embedded within a second matrix, the first and second matrices may be comprised of the same or different materials.

To increase the amount of active compound reaching the target tissue or organ, the final product can comprise a plurality of nanocarriers packed in a single encasing. The nano- or micro-particulate structure of the composition or formulation of the invention, together with the other ingredients, can confer a long-acting, prolonged or sustained effect to the encapsulated CB₁ or CB₂ receptor blocker.

The compositions can form basis for the design of various dosage forms for various applications and modes of administration, for example injectable dosage forms to be administered parenterally, or tablets for oral administration, or powders for inhalation for nasal or pulmonary delivery.

In numerous embodiments, the compositions constitute pharmaceutical compositions in a form suitable for administration to a human or animal subject. The term “pharmaceutical composition” comprises a therapeutically effective amount of a compound of the invention, optionally together with suitable additives such as diluents, preservatives, solubilizers, emulsifiers, adjuvant and/or carriers. The compositions may be liquids or lyophilized or otherwise dried formulations and include diluents of various buffer content (e.g.; Tris-HCL, acetate, phosphate), pH and ionic strength, additives such as albumin or gelatin to prevent absorption to surfaces, detergents (e.g., Tween 20, Tween 80, Pluronic F68, bile acid salts), solubilizing agents (e.g., glycerol, polyethylene glycerol), anti-oxidants (e.g., ascorbic acid, sodium metabisulfite), preservatives (e.g., Thimerosal, benzyl alcohol, parabens), and others.

Pharmaceutical compositions for oral administration can comprise of (a) liquid solutions, such as an effective amount of the compound dissolved in diluents, such as water, saline, or orange juice; (b) capsules, sachets, tablets, lozenges, and troches, each containing a predetermined amount of the active ingredient, as solids or granules; (c) powders; (d) suspensions in an appropriate liquid; and (c) suitable emulsions or self-emulsifying formulations. Liquid formulations may include diluents, such as water and alcohols, for example, ethanol, benzyl alcohol, and the polyethylene alcohols, either with or without the addition of a pharmaceutically acceptable surfactant, suspending agent, or emulsifying agent. Capsule forms can be of the ordinary hard- or soft-shelled gelatin type containing, for example, surfactants, lubricants, and inert fillers. Tablet forms can include one or more of lactose, sucrose, mannitol, corn starch, potato starch, alginic acid, microcrystalline cellulose, acacia, gelatin, guar gum, colloidal silicon dioxide, croscarmellose sodium talc, magnesium stearate, calcium stearate, zinc stearate, stearic acid, and other excipients, colorants, diluents, buffering agents, disintegrating agents, moistening agents, preservatives, flavoring agents, and pharmacologically compatible carriers. Lozenge forms can comprise the active ingredient in a flavor, usually sucrose and acacia or tragacanth, as well as pastilles comprising the active ingredient in an inert base, such as gelatin and glycerin, or sucrose and acacia, emulsions, gels, and the like containing, in addition to the active ingredient, such carriers as are known in the art.

Pharmaceutical compositions for parenteral administration can include sterile nanoemulsions, aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that include suspending agents, solubilizers, thickening agents, stabilizers, and preservatives.

Pharmaceutical compositions can be administered in a physiologically acceptable diluent in a pharmaceutical carrier, such as a sterile liquid or mixture of liquids, including water, saline, aqueous dextrose and related sugar solutions, an alcohol, such as ethanol, isopropanol, or hexadecyl alcohol, glycols, such as propylene glycol or polyethylene glycol, glycerol ketals, such as 2,2-dimethyl-1,3-dioxolane-4-methanol, ethers, such as poly(ethyleneglycol) 400, an oil, a fatty acid, a fatty acid ester or glyceride, or an acetylated fatty acid glyceride with or without the addition of a pharmaceutically acceptable surfactant, such as a soap or a detergent, suspending agent, such as pectin, carbomers, methylcellulose, hydroxypropylmethylcellulose, or carboxymethylcellulose, or emulsifying agents and other pharmaceutical adjuvants. Oils, which can be used in parenteral formulations include petroleum, animal, vegetable, or synthetic oils. Specific examples of oils include peanut, soybean, sesame, cottonseed, corn, olive, petrolatum, and mineral. Suitable fatty acids for use in parenteral formulations include oleic acid, stearic acid, and isostearic acid.

Pharmaceutical compositions can be made as injectable formulations. The requirements for effective pharmaceutical carriers for injectable compositions are well known to those of ordinary skill in the art. See Pharmaceutics and Pharmacy Practice, J.B. Lippincott Co., Philadelphia, Pa., Banker and Chalmers, eds., pages 238-250 (1982), and ASHP Handbook on Injectable Drugs, Toissel, 4th ed., pages 622-630 (1986).

Thus, in numerous embodiments the pharmaceutical compositions of the invention can be in a form suitable for oral, parenteral, subcutaneous, intravenous, intramuscular or intraperitoneal administration.

In some embodiments the pharmaceutical compositions of the invention can be adapted for oral administration.

In other embodiments, the pharmaceutical compositions of the invention can be adapted for IV (intravenous) or IM (intramuscular) administration.

It is another objective of the invention to provide a series of methods, uses and clinical applications of compositions, and specifically those using the compositions for the prevention, alleviation and treatment of disorders or conditions related to the activity of peripherally restricted CB₁ and CB₂ receptors. One example of clinical manifestation of such disorders and conditions is a metabolic disease (also a metabolic syndrome).

The term “metabolic syndrome” usually denotes a cluster of conditions that occur together and generally increase the heart disease, stroke and type 2 diabetes. These conditions include increased blood pressure, high blood sugar, excess body fat around the waist, and abnormal cholesterol or triglyceride levels. This term further encompasses disorders are obesity, insulin resistance, diabetes, coronary heart disease, liver cirrhosis, fatty liver disease, chronic kidney disease and/or cancer.

Thus, in numerous embodiments the invention can provide compositions and methods for use in preventing, alleviating or treating a metabolic disease.

In further embodiments the invention can provide compositions and methods for use in preventing of alleviating or treating a disorder or a condition selected from obesity, insulin resistance, diabetes, coronary heart disease, liver cirrhosis, fatty liver disease, chronic kidney disease and/or cancer, as per recognized clinical diagnoses.

In further embodiments the invention can provide compositions and methods for use in preventing of alleviating or treating comprising one or more of the following symptoms: a reduction in a body weight, a reduction in a body fat, a reduction in blood pressure (hypertension), a reduction in the serum levels of LDL (low-density) cholesterol, an increase in the serum levels of HDL (high-density) cholesterol, an increase in the serum HDL/LDL cholesterol ratio, an increase in the serum triglycerides.

In certain embodiments the compositions and methods of the invention can be part of a combination therapies administered together or in succession with the convention treatments for these disorders.

In certain embodiments, the the compositions and methods of the invention can be used for in the prevention, alleviation or treatment of a weight gain. The term “weight gain” encompasses herein deviations of at least 5%, 10%, 15%, 20%, 25%. 30%, 35%, 40% or more of the body weight in the normal range, as per height, age, gender and clinical history of the treated individual.

Ultimately, the invention provides a series of compositions and formulations for manufacture of medicaments for preventing, alleviating or treating a group of diseases generally termed as a metabolic disease. And in some embodiments the compositions and formulations of the invention can serve for manufacture of medicaments for preventing, alleviating or treating weight gain.

The term “about” in all its appearances in the text denotes up to a ±10% deviation from the specified values and/or ranges, more specifically, up to ±1%, ±2%, ±3%, ±4%, ±5%, ±6%, ±7%, ±8%, ±9% or ±10% deviation therefrom.

### EXAMPLES

Any method and material similar or equivalent to those described herein can be used in the practice or testing of the present invention. Some embodiments of the invention will be now described by way of examples with reference to respective figures.

### Materials and Methods

Synthesis and characterization of BB8 analogs: The hydrochloric acid salt of Building Block 8 (BB8), (R,S)-2-(2-chlorophenyl)-3-(4-chlorophenyl)-5,6,7,8-tetrahydro-2H-oxepino[3,2-c]pyrazol-8-aminium chloride, was prepared according to published procedures (Dow et al. ACS Med. Chem. Lett. 2012, 3, 397-401).

Procedure A: For the reactions of BB8 with carboxylic acid derivatives, the relevant carboxylic acid was suspended in DCM and triethylamine (TEA) and hydroxybenzotriazole (HOBt) were added. The solution was cooled in ice bath, adding N-(3-Dimethylaminopropyl)-N′-ethylcarbodiimide hydrochloride (EDC). After activation (as per each compound below), (R,S)-2-(2-chlorophenyl)-3-(4-chlorophenyl)-5,6,7,8-tetrahydro-2H-oxepino[3,2-c]pyrazol-8-aminium chloride (BB8) was added and the reaction was stirred overnight. The reaction was followed by TLC (2% MeOH in DCM) to ensure completion. The reaction was taken up in additional DCM (50-100 mL) and washed with saturated sodium bicarbonate solution and brine. The organic phase was dried over anhydrous sodium sulfate, filtered, and evaporated to give the crude product. Purification of the product was achieved by flash purification on silica or RP silica as indicated for each compound. Final compounds were analyzed and characterized by HPLC-MS and 1H NMR.

Procedure A was used for the preparation of the following compounds:

N-[2-(2-chlorophenyl)-3-(4-chlorophenyl)-5,6,7,8-tetrahydrooxepino[3,2-c]pyrazol-8-yl]-1H-1,2,4-triazole-3-carboxamide (BNS801): was prepared from 1H-1,2,4-triazole-3-carboxylic acid (83 mg, 0.73 mmol) TEA (340 μL, 7.3 mmol). HOBt (112 mg, 0.73 mmol) and EDC (140 mg, 0.73 mmol). BB8 (100 mg, 0.24 mmol) was added after 2 hr activation. The product was purified by RP C18 flash purification (0.49 mg, 43% yield).

N-[2-(2-chlorophenyl)-3-(4-chlorophenyl)-5,6,7,8-tetrahydrooxepino[3,2-c]pyrazol-8-yl]-1H-pyrazole-3-carboxamide (BNS802): was prepared from 1H-pyrazole-3-carboxylic acid (123 mg, 1.10 mmol) TEA (509 μL, 3.65 mmol). HOBt (168 mg, 1.10 mmol) and EDC (210 mg, 1.10 mmol). BB8 (150 mg, 0.37 mmol) was added after 2 hr activation. The product was purified by silica flash purification (121 mg, 71% yield).

N-[2-(2-chlorophenyl)-3-(4-chlorophenyl)-5,6,7,8-tetrahydrooxepino[3,2-c]pyrazol-8-yl]pyridine-3-carboxamide (BNS803): was prepared from nicotinic acid (135 mg, 1.10 mmol) TEA (509 μL, 3.65 mmol). HOBt (168 mg, 1.10 mmol) and EDC (210 mg, 1.10 mmol). BB8 (150 mg, 0.37 mmol) was added after 2 hr activation. The product was purified by RP C18 flash purification (151 mg, 86% yield).

2-(4-chlorophenoxy)-N-[2-(2-chlorophenyl)-3-(4-chlorophenyl)-5,6,7,8-tetrahydrooxepino[3,2-c]pyrazol-8-yl]-2-methyl-propanamide (BNS804): was prepared from Clofibric acid (235 mg, 1.10 mmol) TEA (509 μL, 3.65 mmol). HOBt (168 mg, 1.10 mmol) and EDC (210 mg, 1.10 mmol). BB8 (150 mg, 0.37 mmol) was added after 2 hr activation. The product was purified by RP C18 flash purification (141 mg, 68% yield).

N-[2-(2-chlorophenyl)-3-(4-chlorophenyl)-5,6,7,8-tetrahydrooxepino[3,2-c]pyrazol-8-yl]pyrazine-2-carboxamide (BNS805): was prepared from pyrazine-2-carboxylic acid (136 mg, 1.10 mmol) TEA (509 μL, 3.65 mmol). HOBt (168 mg, 1.10 mmol) and EDC (210 mg, 1.10 mmol). BB8 (150 mg, 0.37 mmol) was added after 2 hr activation. The product was purified by RP C18 flash purification (130 mg, 74% yield).

N-[2-(2-chlorophenyl)-3-(4-chlorophenyl)-5,6,7,8-tetrahydrooxepino[3,2-c]pyrazol-8-yl]-1H-imidazole-2-carboxamide (BNS806): was prepared from 1H-imidazole-2-carboxylic acid (123 mg, 1.10 mmol) TEA (509 μL, 3.65 mmol). HOBt (168 mg, 1.10 mmol) and EDC (210 mg, 1.10 mmol). BB8 (150 mg, 0.37 mmol) was added after 2 hr activation. The product was purified by RP C18 flash purification (76 mg, 44% yield).

N-[2-(2-chlorophenyl)-3-(4-chlorophenyl)-5,6,7,8-tetrahydrooxepino[3,2-c]pyrazol-8-yl]pyridine-2-carboxamide (BNS810): was prepared from picolinic acid (135 mg, 1.10 mmol) TEA (509 μL, 3.65 mmol). HOBt (168 mg, 1.10 mmol) and EDC (210 mg, 1.10 mmol). BB8 (150 mg, 0.37 mmol) was added after 2 hr activation. The product was purified by RP C18 flash purification (160 mg, 91% yield).

N-[2-(2-chlorophenyl)-3-(4-chlorophenyl)-5,6,7,8-tetrahydrooxepino[3,2-c]pyrazol-8-yl]pyrimidine-4-carboxamide (BNS811): was prepared from pyrimidine-4-carboxylic acid (136 mg, 1.10 mmol) TEA (509 μL, 3.65 mmol). HOBt (168 mg, 1.10 mmol) and EDC (210 mg, 1.10 mmol). BB8 (150 mg, 0.37 mmol) was added after 2 hr activation. The product was purified by RP C18 flash purification (120 mg, 68% yield).

N-[2-(2-chlorophenyl)-3-(4-chlorophenyl)-5,6,7,8-tetrahydrooxepino[3,2-c]pyrazol-8-yl]pyridine-4-carboxamide (BNS812): was prepared from isonicotinic acid (135 mg, 1.10 mmol) TEA (509 μL, 3.65 mmol). HOBt (168 mg, 1.10 mmol) and EDC (210 mg, 1.10 mmol). BB8 (150 mg, 0.37 mmol) was added after 2 hr activation. The product was purified by RP C18 flash purification (115 mg, 66% yield).

N-(2-(2-chlorophenyl)-3-(4-chlorophenyl)-5,6,7,8-tetrahydro-2H-oxepino[3,2-c]pyrazol-8-yl)-1-(2,2,2-trifluoroethyl)piperidine-4-carboxamide (BNS814): was prepared from 1-(2,2,2-trifluoroethyl)piperidine-4-carboxylic acid (123 mg, 0.58 mmol) TEA (244 μL, 1.75 mmol). HOBt (90 mg, 0.58 mmol) and EDC (112 mg, 0.58 mmol). BB8 (0.20 mg, 0.49 mmol) was added after 5 min activation. The product was purified by RP C18 flash purification (205 mg, 74% yield).

N-[2-(2-chlorophenyl)-3-(4-chlorophenyl)-5,6,7,8-tetrahydrooxepino[3,2-c]pyrazol-8-yl]-2-(methylamino)acetamide (BNS819): BNS819 was prepared from Boc-sarcosine (97 mg, 0.51 mmol) TEA (271 μL, 1.95 mmol). HOBt (78 mg, 0.51 mmol) and EDC (98 mg, 0.51 mmol). BB8 (200 mg, 0.49 mmol) was added after 5 min activation. The product was purified by RP C18 flash purification (237 mg, 0.43 mmol). Boc protection was removed with neat TFA (3 mL) for 2 hours. The TFA was evaporated, the reaction was taken up in DCM (50-100 mL) and washed with saturated sodium bicarbonate solution and brine. The organic phase was dried over anhydrous sodium sulfate, filtered, and evaporated to give the final product. (185 mg, 85% yield).

(2R)—N-[2-(2-chlorophenyl)-3-(4-chlorophenyl)-5,6,7,8-tetrahydrooxepino[3,2-c]pyrazol-8-yl]pyrrolidine-2-carboxamide (BNS820): was prepared from Boc-D-proline (110 mg, 0.51 mmol) TEA (271 μL, 1.95 mmol), HOBt (78 mg, 0.51 mmol) and EDC (98 mg, 0.51 mmol). BB8 (200 mg, 0.49 mmol) was added after 5 min activation. The product was purified by RP C18 flash purification (230 mg, 0.40 mmol). Boc protection was removed with neat TFA (3 mL) for 2 hr. The TFA was evaporated, the reaction was taken up in DCM (50-100 mL) and washed with saturated sodium bicarbonate solution and brine. The organic phase was dried over anhydrous sodium sulfate, filtered, and evaporated to give the final product. (168 mg, 73% yield).

(2S)—N-[2-(2-chlorophenyl)-3-(4-chlorophenyl)-5,6,7,8-tetrahydrooxepino[3,2-c]pyrazol-8-yl]pyrrolidine-2-carboxamide (BNS821): was prepared from Boc-L-proline (110 mg, 0.51 mmol) TEA (271 μL, 1.95 mmol), HOBt (78 mg, 0.51 mmol) and EDC (98 mg, 0.51 mmol). BB8 (200 mg, 0.49 mmol) was added after 5-minute activation. The product was purified by RP C18 flash purification (245 mg, 0.45 mmol). Boc protection was removed with neat TFA (3 mL) for 2 hr. The TFA was evaporated, the reaction was taken up in DCM (50-100 mL) and washed with saturated sodium bicarbonate solution and brine. The organic phase was dried over anhydrous sodium sulfate, filtered, and evaporated to give the final product. (171 mg, 79% yield).

N-[2-(2-chlorophenyl)-3-(4-chlorophenyl)-5,6,7,8-tetrahydrooxepino[3,2-c]pyrazol-8-yl]-5-(2,5-dimethylphenoxy)-2,2-dimethyl-pentanamide (BNS822): was prepared from Gemfibrozil (64 mg, 0.26 mmol) TEA (136 μL, 0.97 mmol), HOBt (39 mg, 0.26 mmol) and EDC (49 mg, 0.26 mmol). BB8 (100 mg, 0.24 mmol) was added after 5 min activation. The product was purified by RP C18 flash purification (124 mg, 84% yield).

2-[4-(4-chlorobenzoyl)phenoxy]-N-[2-(2-chlorophenyl)-3-(4-chlorophenyl)-5,6,7,8-tetrahydrooxepino[3,2-c]pyrazol-8-yl]-2-methyl-propanamide (BNS823): was prepared from Fenofibric acid (81 mg, 0.26 mmol) TEA (136 μL, 0.97 mmol), HOBt (39 mg, 0.26 mmol) and EDC (49 mg, 0.26 mmol). BB8 (100 mg, 0.24 mmol) was added after 5 min activation. The product was purified by RP C18 flash purification (134 mg, 82% yield).

N-(2-(2-chlorophenyl)-3-(4-chlorophenyl)-5,6,7,8-tetrahydro-2H-oxepino[3,2-c]pyrazol-8-yl)piperidine-4-carboxamide (BB8-INT-1): was prepared from 1-Boc-piperidine-4-carboxylic acid (205 mg, 0.89 mmol) TEA (475 μL, 3.41 mmol), HOBt (137 mg, 0.89 mmol) and EDC (171 mg, 0.89 mmol). BB8 (350 mg, 0.85 mmol) was added after 5 min activation. The product was purified by RP C18 flash purification (471 mg, 0.80 mmol). Boc protection was removed with neat TFA (3 mL) for 2 hours. The TFA was evaporated, the reaction was taken up in DCM (50-100 mL) and washed with saturated sodium bicarbonate solution and brine. The organic phase was dried over anhydrous sodium sulfate, filtered, and evaporated to give the final product. (384 mg, 93% yield).

N-[2-(2-chlorophenyl)-3-(4-chlorophenyl)-5,6,7,8-tetrahydrooxepino[3,2-c]pyrazol-8-yl]-1-(pyridine-3-carbonyl)piperidine-4-carboxamide (BNS817): was prepared from nicotinic acid (72 mg, 0.59 mmol) TEA (245 μL, 1.76 mmol), HOBt (90 mg, 0.59 mmol) and EDC (112 mg, 0.59 mmol). BB8-INT-1 (95 mg, 0.2 mmol) was added after 2 hr activation. The product was purified by RP C18 flash purification (101 mg, 87% yield).

N-[2-(2-chlorophenyl)-3-(4-chlorophenyl)-5,6,7,8-tetrahydrooxepino[3,2-c]pyrazol-8-yl]-1-(2-methylsulfonylacetyl)piperidine-4-carboxamide (BNS824): was prepared from 2-(methylsulfonyl)acetic acid (25 mg, 0.18 mmol) TEA (73 μL, 0.53 mmol), HOBt (28 mg, 0.18 mmol) and EDC (35 mg, 0.18 mmol). BB8-INT-1 (85 mg, 0.18 mmol) was added after 5 min activation. The product was purified by RP C18 flash purification (90 mg, 85% yield).

Procedure B: For the reactions of BB8 with sulfonyl chloride and acetyl chloride derivatives, (R,S)-2-(2-chlorophenyl)-3-(4-chlorophenyl)-5,6,7,8-tetrahydro-2H-oxepino[3,2-c]pyrazol-8-aminium chloride (BB8) was dissolved in DCM (0.1 M). The solution was cooled in an ice bath and the appropriate sulfonyl chloride (1.2 equiv.) and TEA (1.2 equiv.) were added. The reaction was monitored by TLC (2% MeOH in DCM) till completion. The reaction was taken up in additional DCM (50-100 mL) and washed with saturated sodium bicarbonate solution and brine. The organic phase was dried over anhydrous sodium sulfate, filtered, and evaporated to give the crude product. Purification of the product was achieved by flash purification on silica or RP silica as per each compound below. Final compounds were analyzed and characterized by HPLC-MS and 1H NMR.

Procedure A was used for the preparation of the following compounds:

N-[2-(2-chlorophenyl)-3-(4-chlorophenyl)-5,6,7,8-tetrahydrooxepino[3,2-c]pyrazol-8-yl]-4-methyl-benzenesulfonamide (BNS807): was prepared from BB8 (150 mg, 0.37 mmol). Tosyl chloride (104 mg, 0.55 mmol), and TEA (153 μL, 1.1 mmol). The product was purified by RP C18 flash purification (190 mg, 98% yield).

4-chloro-N-[2-(2-chlorophenyl)-3-(4-chlorophenyl)-5,6,7,8-tetrahydrooxepino[3,2-c]pyrazol-8-yl]benzenesulfonamide (BNS808): was prepared from BB8 (150 mg, 0.37 mmol). 4-chlorobenzenesulfonyl chloride (116 mg, 0.55 mmol), and TEA (153 μL, 1.1 mmol). The product was purified by RP C18 flash purification (190 mg, 98% yield).

N-[2-(2-chlorophenyl)-3-(4-chlorophenyl)-5,6,7,8-tetrahydrooxepino[3,2-c]pyrazol-8-yl]methanesulfonamide (BNS809): was prepared from BB8 (150 mg. 0.37 mmol). Mesyl chloride (42 μL, 0.55 mmol), and TEA (153 μL, 1.1 mmol). The product was purified by RP C18 flash purification (160 mg, 97% yield).

1-[2-(2-chlorophenyl)-3-(4-chlorophenyl)-5,6,7,8-tetrahydrooxepino[3,2-c]pyrazol-8-yl]-3-methyl-urea (BNS813): was prepared from BB8 (150 mg, 0.37 mmol), methylcarbamic chloride (51 mg, 0.55 mmol), and TEA (153 μL, 1.1 mmol). The product was purified by silica flash purification (156 mg, 99% yield).

N-[2-(2-chlorophenyl)-3-(4-chlorophenyl)-5,6,7,8-tetrahydrooxepino[3,2-c]pyrazol-8-yl]-1-methylsulfonyl-piperidine-4-carboxamide (BNS815): was prepared from BB8-INT-1 (90 mg, 0.19 mmol). Mesyl chloride (22 μL, 0.28 mmol), and TEA (39 μL, 0.28 mmol). The product was purified by silica flash purification (94 mg, 90% yield).

Ethyl-4-((2-(2-chlorophenyl)-3-(4-chlorophenyl)-5,6,7,8-tetrahydro-2H-oxepino[3,2-c]pyrazol-8-yl)carbamoyl)piperidine-1-carboxylate (BNS816): was prepared from BB8-INT-1 (90 mg, 0.19 mmol). Ethyl chloroformate (26 μL, 0.28 mmol), and TEA (39 μL, 0.28 mmol). The product was purified by silica flash purification (98 mg, 95% yield).

N4-[2-(2-chlorophenyl)-3-(4-chlorophenyl)-5,6,7,8-tetrahydrooxepino[3,2-c]pyrazol-8-yl]-N1-methyl-piperidine-1,4-dicarboxamide (BNS818): was prepared from BB8-INT-1 (100 mg, 0.21 mmol), methylcarbamic chloride (29 mg, 0.31 mmol), and TEA (43 μL, 0.31 mmol). The product was purified by RP C18 flash purification (103 mg, 92% yield).

N-[2-(2-chlorophenyl)-3-(4-chlorophenyl)-5,6,7,8-tetrahydrooxepino[3,2-c]pyrazol-8-yl]-1-(4-chlorophenyl)sulfonyl-piperidine-4-carboxamide (BNS825): was prepared from BB8-INT-1 (85 mg, 0.18 mmol). 4-chlorobenzenesulfonyl chloride (55 mg, 0.26 mmol), and TEA (49 μL, 0.35 mmol). The product was purified by RP C18 flash purification (91 mg, 79% yield).

2-((4-chloro-N-methylphenyl)sulfonamido)-N-(2-(2-chlorophenyl)-3-(4-chlorophenyl)-5,6,7,8-tetrahydro-2H-oxepino[3,2-c]pyrazol-8-yl)acetamide (BNS826): was prepared from BNS819 (141 mg, 0.31 mmol). 4-chlorobenzenesulfonyl chloride (80 mg, 0.38 mmol), and TEA (88 μL, 0.63 mmol). The product was purified by RP C18 flash purification (167 mg, 85% yield).

(2R)—N-(2-(2-chlorophenyl)-3-(4-chlorophenyl)-5,6,7,8-tetrahydro-2H-oxepino[3,2-c]pyrazol-8-yl)-1-((4-chlorophenyl)sulfonyl)pyrrolidine-2-carboxamide (BNS827): was prepared from BNS820 (148 mg, 0.31 mmol). 4-chlorobenzenesulfonyl chloride (79 mg, 0.38 mmol), and TEA (87 μL, 0.63 mmol). The product was purified by RP C18 flash purification (171 mg, 84% yield).

(2S)—N-(2-(2-chlorophenyl)-3-(4-chlorophenyl)-5,6,7,8-tetrahydro-2H-oxepino[3,2-c]pyrazol-8-yl)-1-((4-chlorophenyl)sulfonyl)pyrrolidine-2-carboxamide (BNS828): was prepared from BNS821 (140 mg, 0.30 mmol). 4-chlorobenzenesulfonyl chloride (75 mg, 0.36 mmol), and TEA (83 μL, 0.59 mmol). The product was purified by RP C18 flash purification (163 mg, 85% yield).

Radioligand binding assays: Binding affinity was determined by a radioligand binding assay. The different BB8-conjugates binding to CB₁ and CB₂ receptors was assessed in competition displacement assays using [³H]CP-55.940 as the radioligand and crude membranes from mouse brain for CB₁ receptor and human kidney cells for CB₂ receptor.

CB₁ receptor binding assay: Mouse brain membranes were used as the source material for CB₁ receptors. The displacement of specifically bound tritiated CP-55.940 from these membranes using a standard filtration assay was used to determine the Ki values for the test compounds. Briefly. 20 μg of protein was incubated for 1 h at 30° C. in the presence of 0.5 nM [³H]CP-55,940 and various concentrations (10⁻⁵M-10⁻¹¹M) of test compound/control, final volume of 1 mL. The incubation was terminated by rapid filtration and washing, and the amount of specifically bound [³H]CP-55,940 was determined. Briefly, membranes with bound [³H]CP-55,940 were separated and washed from free ligand by vacuum filtration, adsorbing the membrane onto a Whatman glass microfiber filter paper (LIFEGENE, Cat #1821271). Finally, the Whatman filter paper with adsorbed membranes was cut and placed in scintillation liquid (Ultima Gold) for 1 h at 25° C. followed by a β counter reading of bound [³H]CP-55,940 radioligand. All data were in triplicates with Ki values determined using GraphPad Prism 7.02 analysis software. Data normalized between 0 and 100% specific binding were plotted against log concentration of test compound, and Ki was extracted using nonlinear regression analysis. In some cases, membranes from human source were used to determine CB₁ receptor binding affinity.

CB₂ receptor binding assay: Human kidney membranes were used as the source material for CB₂ receptors. The displacement of specifically bound tritiated CP-55,940 from these membranes using a standard filtration assay was used to determine the Ki values for the test compounds. Briefly, 1.25 μg of protein was incubated for 1.5 h at 30° C. in the presence of 0.5 nM [³H]CP-55,940 and various concentrations (10⁻⁵M-10⁻¹¹M) of test compound/control, final volume of 1 mL. The incubation was terminated by rapid filtration and washing, and the amount of specifically bound [³H]CP-55,940 was determined. Briefly, membranes with bound [³H]CP-55,940 were separated and washed from free ligand by vacuum filtration, adsorbing the membrane onto a Whatman glass microfiber filter paper (LIFEGENE, Cat #1821271). Finally, the Whatman filter paper with adsorbed membranes was cut and placed in scintillation liquid (Ultima Gold) for 1 h at 25° C. followed by a β counter reading of bound [³H]CP-55,940 radioligand. All data were in triplicates with Ki values determined using GraphPad Prism 7.02 analysis software. Data normalized between 0 and 100% specific binding were plotted against log concentration of test compound, and Ki was extracted using nonlinear regression analysis.

[³⁵S]GTPγS binding assay for CB₁ receptor: The nature of binding (agonist/antagonist/inverse agonist) to CB₁ receptor was determined by [³⁵S]GTPγS binding assay. For CB₁ receptor-mouse brains were dissected and P2 membranes prepared and resuspended at ˜2 μg protein/μL in 1 ml assay buffer (50 mM Tris HCl, 9 mM MgCl2, 0.2 mM EDTA, 150 mM NaCl; pH 7.4). Ligand-stimulated [³⁵S]GTPγS binding was assayed as described previously (Tam et al., JCI 2010). Briefly, membranes (10 μg protein) were incubated in assay buffer containing 100 μM GDP, 0.05 nM [³⁵S]GTPγS, test compounds at various concentrations (CP55940/Rimonabant as controls and test compounds for BB8 conjugates), and 1.4 mg/mL fatty acid-free BSA in siliconized glass tubes. Membranes with bound ligand ([³⁵S]GTPγS) were separated from free ligand by vacuum filtration and were analyzed using β counter as described above. Non-specific binding was determined using 10 μM GTPS (cold GTP). Basal binding was assayed in the absence of the tested compound and in the presence of GDP.

MDR1-MDCK II cell permeation assay: MDR1-MDCK II cells (obtained from Piet Borst at the Netherlands Cancer Institute) were seeded onto polyethylene membranes (PET) in 96-well insert systems at 2.5×105 cells/mL until to 4-7 days for confluent cell monolayer formation. Test and reference compounds were diluted with transport buffer (HBSS with 10 mM Hepes, pH 7.4) from stock solution to a concentration of 2 μM (DMSO<1%) and applied to the apical or basolateral side of the cell monolayer. Permeation of the test compounds from A to B direction or B to A direction was determined in duplicate with/without P-gp inhibitor (GF120918, 10 μM). Digoxin was tested at 10 μM in the presence or absence of 10 μM GF120918 bi-directionally as well, while nadolol and metoprolol were tested at 2 μM in the absence of GF120918 in A to B direction in duplicate. The plate was incubated for 2.5 hours in CO2 incubator at 37±1° C., with 5% CO2 at saturated humidity without shaking. In addition, the efflux ratio of each compound was also determined. Test and reference compounds were quantified by LC-MS/MS analysis based on the peak area ratio of analyte/IS.

Mini-AMES assay: The mutagenic potential of the test articles, or its metabolites, was evaluated by measuring its ability to induce reverse mutations at selected loci of bacteria Salmonella typhimurium (TA98, TA100) and in both the presence and absence of microsomal enzymes (S9). The test strains were prepared from frozen working stocks. 10 μL frozen working stock adding in 5 mL nutrient broth were incubated with 220 rpm shaking at 37±2° C. for 10 hours until an optical density (at 650 nm) of 0.6˜0.8 were reached. The overnight culture was used for the mutagenicity test. Test articles stock solutions were prepared at 50 mg/mL in DMSO. Sub-doses were prepared by dilution in DMSO from the stock immediately prior to use. If the test article was not soluble at 50 mg/mL, the concentration was reduced to the lowest soluble concentration below 50 mg/ml. DMSO was used as negative control, and the positive controls are described in Table 1 below.

TABLE 1
Conditions used in the mini-AMES assays

| | S9 (microsomal | | Concentration |
|---|---|---|---|
| Strain | enzymes) | Positive control | (μg/well) |
| TA98 | + | 2-Aminoanthracene | 2 |
| | − | 2-Nitroflurene | 4 |
| TA100 | + | 2-Aminoanthracene | 2 |
| | − | MNNG | 1 |

The followings were added in order into a test tube for each concentration: (a) 1600 μL of Top Agar (b) 80 μL of drug or controls (c) 400 μL of S9 mix or PBS buffer (d) 80 μL of overnight culture. Vortexed and dispensed 540 μL/well using a disposable pipette. Plates were incubated at 37±2° C. for approximately 48˜72 hr.

Tissue distribution and pharmacokinetics: Tissue levels of antagonist: 3 male C57BL/6J mice (7-9 weeks, PO group) were used in this study. Mice were fasted prior to administration of test article and had access to food 4 hours post dosing. Appropriate amount of test article was accurately weighted and mixed with the appropriate volume of vehicle to get a clear oral solution. The formulation was prepared on the day of dosing, and mice were dosed via oral gavage up to 4 hr after formulation was prepared. After 1 hour, mice were scarified and about 200 μL blood was collected from cardiac puncture followed by plasma preparation. Brain and liver were removed and further processed. Dose formulation and sample analysis was performed by LC-MS/MS method.

Pharmacokinetics: Male C57BL/6J mice (7-9 weeks, N=3 in each group) were used in this study. Mice were fasted at least 12 hr prior to administration of test article and have access to food ad libitum 4 hr post dosing. Appropriate amount of test article was accurately weighted and mixed with the appropriate volume of vehicle to get a clear oral solution. The formulations were prepared on the day of dosing, and mice were dosed via either orally gavage or intravenous injection, up to 4 hr after formulations were prepared. About 30 μL blood per time point was collected from the saphenous vein followed by plasma preparation. Dose formulation and sample analysis was performed by LC-MS/MS method. Plasma concentration versus time data was analyzed by non-compartmental method.

In vivo efficacy study in Diet Induced Obesity (DIO) model: Male C57BL/6J mice were kept on a high fat diet for 24 weeks. Then, mice were treated with the different treatments of test articles for 3 weeks (Daily PO treatment). Thereafter, metabolic assessment was performed in order to evaluate the efficacy of the treatment. The efficacy was evaluated by measurements of the following parameters:
1. Body weight and food intake,
2. Total energy expenditure (TEE) and respiratory quotient (RQ).
3. Fat and Carbohydrate oxidation (FO and CHO, respectively),
4. Total ambulation,
5. Glucose homeostasis,
6. Hepatic Steatosis and Liver Injury,
7. Lipid profile,
8. Kidney function.

Animals: The experimental protocol used is approved by the Institutional Animal Care and Use Committee of the Hebrew University. Male, 25 weeks old, C57BL/6J mice were purchased from Jackson laboratories. Mice were maintained under a 12-h light/dark cycle and fed ad libitum. To maintain diet-induced obesity, C57BL/6J mice were fed with high-fat diet (HFD) (60% of calories from fat, 20% from protein, and 20% from carbohydrates; Research Diet, D12492) for overall 30 weeks.

HFD-fed obese mice received vehicle/test article daily for 3 weeks by PO administration (using a gavage). Body weight and food intake were monitored daily. Total body fat and lean masses were determined by EchoMRI-100H™ (Echo Medical Systems LLC, Houston, TX, USA). 24 h urine was collected 2-4 days before euthanasia using mouse metabolic cages (CCS2000 Chiller System, Hatteras Instruments, NC, USA). At the end of the observation period, mice were euthanized by a cervical dislocation under anesthesia, the kidneys, liver, brain, spleen, fat, and pancreas were removed, and samples were fixed in buffered 4% formalin (for histopathological analysis) or snap frozen (for biochemistry analysis). Trunk blood was collected for determining the biochemical parameters.

Multi-parameter metabolic assessment: Metabolic profile of the mice was assessed by using the Promethion High-Definition Behavioral Phenotyping System

(Sable Instruments, Inc., Las Vegas, NV, USA). Data acquisition and instrument control were performed using MetaScreen software version 2.2.18.0, and the obtained raw data were processed using ExpeData version 1.8.4 using an analysis script detailing all aspects of data transformation. Mice with free access to food and water were subjected to a standard 12 hr light/12 hr dark cycle, which consisted of 48 hr acclimation period followed by 24 hr of sampling. Respiratory gases were measured by using the GA-3 gas analyzer (Sable Systems, Inc., Las Vegas, NV, USA) using a pull-mode, negative-pressure system. Air flow was measured and controlled by FR-8 (Sable Systems, Inc., Las Vegas, NV, USA), with a set flow rate of 2000 mL/min. Water vapor was continuously measured and its dilution effect on O2 and CO2 was mathematically compensated. Effective mass was calculated by [body mass]0.75. Fat oxidation (FO) and carbohydrate oxidation (CHO) were calculated as FO=1.69×VO₂−1.69×VCO₂ and CHO=4.57×VCO₂−3.23×VO₂ and expressed as g/d/kg^{eff.Mass}.

Locomotor activity: Locomotor activity was quantified by the number of disruptions of infrared XYZ beam arrays with a beam spacing of 0.25 cm in the Promethion High-Definition Behavioral Phenotyping System (Sable Instruments, Inc., Las Vegas, NV, USA).

Elevated plus-maze: Anxiety-related behaviors were assessed using the EPM test as reported previously. Animals were placed on the 5×5 cm central platform of an apparatus from which four arms, 30 cm×5 cm extended. Two of the arms (the closed arms) are enclosed within 15 cm high walls, and the two other arms (the open arms) have 1 cm high rims. The whole maze is elevated 75 cm above the ground. During the 6 min test time, the number of entries to each arm type (closed or open arms, frequencies) and the time spent in each type of arm (closed or open arms, duration) during the test were measured.

Catalepsy test: Catalepsy was assayed using the bar test. Briefly, mice were removed from their home cage, and their forepaws were placed on a horizontal bar, 0.5 cm in diameter, positioned 4 cm above the bench surface. Vehicle-treated mice routinely let go of the bar within 2 sec. Cataleptic behavior was defined as the time the animals remained motionless holding on to the bar, with an arbitrary cutoff of 30 sec. The antagonists were given 30 min before the IP injection of 3 mg/kg WIN55,212. The test was performed 60 min after agonist administration.

Glucose tolerance (ipGTT) test and insulin sensitivity tests (ipIST): Mice that fasted overnight were injected with glucose (1.5 g/kg, IP), followed by a tail blood collection at 0, 15, 30, 45, 60, 90, 120 min. Blood glucose levels were determined using the Elite glucometer (Bayer, Pittsburgh, PA). Two days later, mice were fasted for 6 hr before receiving insulin (0.75 U/kg, IP; Eli Lilly, DC, USA or Actrapid® vial, novo nordisk A/S, Denmark), and blood glucose levels were determined at the same intervals.

Blood and urine biochemistry: Serum and urine levels of creatinine and glucose as well as serum levels of ALT, AST, HDL, LDL, TG, and cholesterol were determined by using the Cobas C-111 chemistry analyzer (Roche, Switzerland). Creatinine clearance was calculated using urine and serum creatinine levels (CCr mL/h=Urine creatinine mg/dL×Urine volume/Serum creatinine mg/dL×24 hr). Fasting blood glucose was measured using the Elite glucometer (Bayer, Pittsburgh, PA).

Histopathological Analyses: 5 μm paraffin-embedded liver sections from 3 animals per group were stained with hematoxylin-eosin staining. Liver images were captured with a Zeiss AxioCam ICc5 color camera mounted on a Zeiss Axio Scope.A1 light microscope and taken from 10 random 40× and 10× fields of each animal.

Statistical analysis: Statistical analysis was performed using GraphPad Prism software version 8. Ordinary t-test was used to determine the difference between Vehicle and treatment group. Differences were considered significant if P<0.05.

Experimental Findings

1. Preparation of Specific Formulations

The BNS808 formulation (0.1% w/w BB8-08 conjugate) was prepared from the ingredients in Table 2 by the process with the main steps of: mixing the ingredients in a vial (20 mL) at 1400 rpm, 35° C. for 15 min until obtaining a clear solution.

TABLE 2
BNS808 formulation (0.1% w/w)

| Ingredients | % w/w | Amount | |
|---|---|---|---|
| BB8-08 | 0.1 | 5 | mg |
| Capryol 90 | 25 | 1.25 | g |
| Cremophor EL | 25.73 | 1.29 | g |
| Propylene glycol | 12.87 | 0.64 | g |
| MCT | 28.51 | 1.43 | g |
| Ethanol | 7.89 | 0.39 g (0.5 ml) | |
| Total | 100 | 5 | g |

The BNS822 formulation (0.8% w/w BB8-22 conjugate; 8 mg/ml) was prepared from the ingredients in Table 3 by the process with the main steps of: mixing Cremophor RH 40, PEG 400, propylene glycol, tripropionin in a step-wise manner while mixing at 40° C. until obtaining a clear solution (SEDDs oil-based vehicle), and adding the BB8-22 conjugate to the vehicle the while stirring at 1400 rpm at 55° C. for 60-90 min until completely dissolved.

TABLE 3
BNS822 formulation (0.8% w/w)

| Ingredients | % w/w | Amount | |
|---|---|---|---|
| BB8-22 | 0.8 | 32 | mg |
| Cremophor RH 40 | 28 | 1400 | mg |
| PEG 400 | 28 | 1400 | mg |
| Propylene glycol | 28 | 1400 | mg |
| Tripropionin | 16 | 800 | mg |

2. In Vitro Binding to CB₁ and CB₂ Receptors

The BB8 conjugated (BNS) compounds were tested for in vitro binding activity to CB₁ and CB₂ receptors. The results are shown in Table 4. Most of the tested conjugates showed good affinity to the CB₁ receptor (mouse/human), with Ki values in the nanomolar range. Two compounds, BNS808 and BNS822 demonstrated high potency against the CB₁ receptor. All the tested compounds proved to be antagonist/inverse agonist to the CB₁ receptor in the GTPγ[³⁵S] binding assay, as expected. Analysis of the CB₂ receptor binding affinity demonstrated that all the tested BB8 conjugates are selective CB₁ inhibitors.

TABLE 4
In vitro CB₁ and CB₂ R binding of the BNS compounds

| | | | | | | mCB₁R | |
|---|---|---|---|---|---|---|---|
| | | | | | CB₁R Ki | GTPγ[³⁵S] | hCB₂R Ki |
| ID # | X (Formula (I)) | X | MW | PSA (Å2) | (nM) | (activity) | (nM) |
| BNS801 | 1H-1,2,4-triazole- 3-carbonyl | 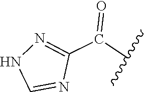 [SMILES: *C(=O)c1nc[nH]n1] | 469 | 98 | 805.2 (m) 231.6 (h) | | |
| BNS802 | 1H-pyrazole- 3-carbonyl | 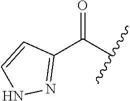 [SMILES: *C(=O)c1cc[nH]n1] | 468 | 85 | ND | | |
| BNS803 | pyridine-3-carbonyl | 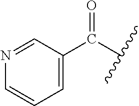 [SMILES: *C(=O)c1cccnc1] | 479 | 69 | 22.35 (m) 6.48 (h) | Inverse agonist | >9111 |
| BNS804 | 2-(4-chlorophenoxy)- 2-methyl-propanyl | 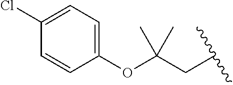 [SMILES: *CC(C)(C)Oc1ccc(Cl)cc1] | 571 | 65 | 1.16 (m) 1.96 (h) | Antagonist | 5697 |
| BNS805 | pyrazine-2-carbonyl | 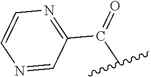 [SMILES: *C(=O)c1cnccn1] | 480 | 82 | 77.75 (m) 8.75 (h) | Inverse agonist | 8339.7 |
| BNS806 | 1H-imidazol-2- carbonyl | 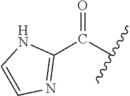 [SMILES: *C(=O)c1ncc[nH]1] | 468 | 85 | 164.8 (m) 23.7 (h) | Antagonist; In. Agonist >[c] | >9111 |
| BNS807 | 4-methyl benzenesulfonyl | 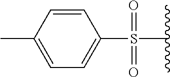 [SMILES: *S(=O)(=O)c1ccc(C)cc1] | 528 | 73 | 10 (m) 1.4 (h) | Inverse agonist | 1650 |
| BNS808 | 4-chloro benzenesulfonyl | 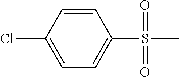 | 549 | 73 | 0.6 (m) 0.7 (h) | Antagonist | 1930.7 |
| BNS809 | methanesulfonyl | 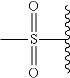 [SMILES: *S(C)(=O)=O] | 452 | 73 | 33.78 (m) 7.7 (h) | Antagonist | >9111 |
| BNS810 | pyridine-2-carbonyl | 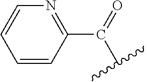 [SMILES: *C(=O)c1ccccn1] | 479 | 69 | 64.47 (m) | Antagonist | 988 |
| BNS811 | pyrimidine-4-carbonyl | 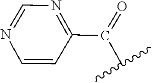 [SMILES: *C(=O)c1ccncn1] | 480 | 82 | 65.58 (m) | Antagonist | 963 |
| BNS812 | pyridine-4-carbonyl | 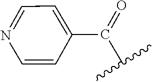 [SMILES: *C(=O)c1ccncc1] | 479 | 69 | 16.26 (m) | Antagonist | 1300 |
| BNS813 | methylcarbamoyl | 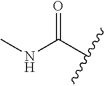 [SMILES: *C(=O)NC] | 431 | 68 | 1130 (m) | | |
| BNS814 | 1-(2,2,2-trifluoroethyl) piperidine-4-carbonyl | 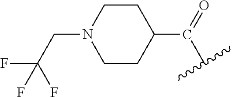 [SMILES: *C(=O)C1CCN(CC(F)(F)F)CC1] | 567 | 59 | | | |
| BNS815 | 1-methylsulfonyl piperidine-4-carbonyl | 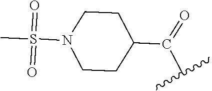 [SMILES: *C(=O)C1CCN(S(C)(=O)=O)CC1] | 563 | 93 | 17.96 (m) | Antagonist | 6312 |
| BNS816 | 1-(ethoxycarbonyl) piperidine-4-carbonyl | 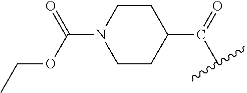 [SMILES: *C(=O)C1CCN(C(=O)OCC)CC1] | 557 | 86 | 8.93 (m) | Inverse Agonist | 1531.7 |
| BNS817 | 1-(pyridine-3-carbonyl) piperidine-4-carbonyl | 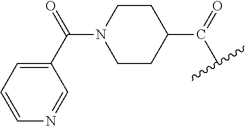 [SMILES: *C(=O)C1CCN(C(=O)c2cccnc2)CC1] | 590 | 89 | 290.4 (m) | Inverse Agonist | No binding |
| BNS818 | 1-(methylcarbamoyl) piperidine-4-carbonyl | 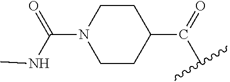 [SMILES: *C(=O)C1CCN(C(=O)NC)CC1] | 542 | 88 | ND | | |
| BNS819 | 2-(methylamino)acetyl | 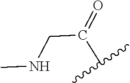 [SMILES: *C(=O)CNC] | 445 | 68 | 922.3 (m) | | |
| BNS820 | (2R)-pyrrolidine-2- carbonyl | 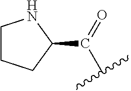 [SMILES: *C(=O)[C@H]1CCCN1] | 471 | 68 | 877.3 (m) | | |
| BNS821 | (2S)-pyrrolidine-2- carbonyl | 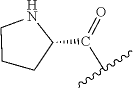 [SMILES: *C(=O)[C@@H]1CCCN1] | 471 | 68 | 1400 (m) | | |
| BNS822 | 5-(2,5- dimethylphenoxy)- 2,2-dimethylpentanoyl | 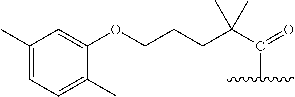 [SMILES: *C(=O)C(C)(C)CCCOc1cc(C)ccc1C] | 607 | 65 | 1.4 (m) | Antagonist | No binding |
| BNS823 | 2-(4-(4-chlorobenzoyl) phenoxy)-2- methylpropanoyl | 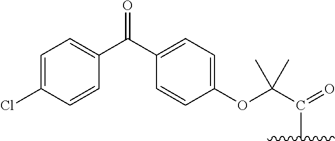 [SMILES: *C(=O)C(C)(C)Oc1ccc(C(=O)c2ccc(Cl)cc2)cc1] | 675 | 82 | 56.7 (m) | Antagonist | No binding |
| BNS824 | 1-(2-(methylsulfonyl) acetyl)piperidine-4- carbonyl | 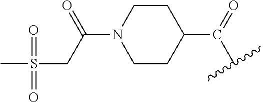 [SMILES: *C(=O)C1CCN(C(=O)CS(C)(=O)=O)CC1] | 605 | 111 | 456.4 (m) | | |
| BNS825 | 1-((4-chlorophenyl) sulfonyl)piperidine-4- carbonyl | 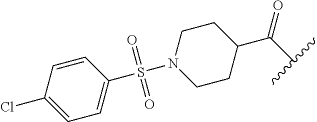 [SMILES: *C(=O)C1CCN(S(=O)(=O)c2ccc(Cl)cc2)CC1] | 660 | 93 | 1.6 (m) | Antagonist | 127 |
| BNS826 | N-((4-chlorophenyl) sulfonyl)-N- methylglycinyl | 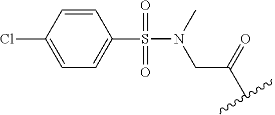 [SMILES: *C(=O)CN(C)S(=O)(=O)c1ccc(Cl)cc1] | 620 | 93 | | | |
| BNS827 | ((4-chlorophenyl) sulfonyl)-D-prolinyl | 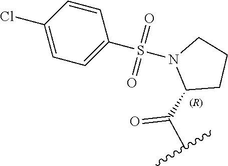 | 646 | 93 | | | |
| BNS828 | ((4-chlorophenyl) sulfonyl)-L-prolinyl | 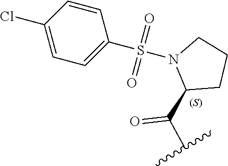 | 646 | 93 | | | |
| Com. 10Q (ref) | 2-(methylsulfonyl) acetyl | 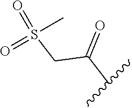 [SMILES: *C(=O)CS(C)(=O)=O] | 494 | 90 | 111.3 (m) | | |

3. In Vitro Bi-Directional Permeability Across MDR1-MDCKII Cells

The in vitro bi-directional permeability across MDR1-MDCKII cells including P-glycoprotein (Pgp, ABCB1) efflux in the presence and absence of a P-gp inhibitor is an indicator as to the tendency of an orally administered compound to penetrate the brain. Table 5 below shows the mean permeability value, the rank Papp and the efflux ratio for each compound. Overall, the results show that selected compounds, BNS808, BNS815, BNS817 and BNS822, and the reference 10Q act as P-gp substrates, which is consistent with peripherally restricted CB₁ receptor blockers.

TABLE 5
In vitro bi-directional permeability of selected BNS compounds

| | | | | MDR1 | |
|---|---|---|---|---|---|
| | MDR1 | | | B:A/A:B | |
| | Pₐₚₚ A:B | Rank | MDR1 | (+P-gp | P-gp |
| ID# | (×10⁻⁶ cm/s) | Papp | B:A/A:B | inhibitor)^{a} | substrate |
| BNS803 | 13.24 | High | 0.77 | 0.63 | No |
| BNS805 | 4.93 | Moderate | 0.82 | 0.55 | Partial |
| BNS808 | 0.26 | Low | 3.95 | 1.88 | Yes |
| BNS815 | 10.09 | High | 1.87 | 0.63 | Yes |
| BNS816 | 6.99 | High | 0.77 | 0.61 | No |
| BNS817 | 8.09 | High | 1.92 | 0.64 | Yes |
| BNS822 | <0.02 | Low | >3.62 | NA | Yes |
| BNS823 | <0.003 | Low | >27.88 | >30.88 | No |
| 10Q | 8.86 | High | 2.6 | 0.69 | Yes |
| Reference | | | | | |

^{a}Permeation of the test compounds from A to B direction or B to A direction was determined in the presence of P-gp inhibitor- GF120918. NA = not available.

4. Findings in the Mini-AMES Assay

The mutagenic potential of two selected compounds, BNS808 and BNS822, was further tested in a mini-AMES assay. The results are summarized in Table 6. Both compounds showed no toxicity at doses range from 31.25˜1000 μg/well on two AMES reference strains, TA98 and TA100. In addition, the compounds showed <2-fold increase in reversion over the negative control, suggesting that the test articles did not induce substantial dose-dependent increases in reversion rates on the AMES strains, with and without S9. In other words, the parent compounds and their metabolites proved to be non-mutagenic.

TABLE 6
BNS808 and BNS822 testing in mini-AMES assay

| | Test | | AMES result | Highest |
|---|---|---|---|---|
| Test article | strain | S9 | (positive/negative) | concentration |
| DMSO | TA98 | yes | negative | NA |
| DMSO | TA98 | no | negative | NA |
| DMSO | TA100 | yes | negative | NA |
| DMSO | TA100 | no | negative | NA |
| 2-Aminoanthracene | TA98 | yes | positive | 2 |
| 2-Nitroflurene | TA98 | no | positive | 4 |
| 2-Aminoanthracene | TA100 | yes | positive | 2 |
| MNNG | TA100 | no | positive | 1 |
| BNS808 | TA98 | yes | negative | 1000 |
| BNS808 | TA98 | no | negative | 1000 |
| BNS808 | TA100 | yes | negative | 1000 |
| BNS808 | TA100 | no | negative | 1000 |
| BNS822 | TA98 | yes | negative | 1000 |
| BNS822 | TA98 | no | negative | 1000 |
| BNS822 | TA100 | yes | negative | 1000 |
| BNS822 | TA100 | no | negative | 1000 |

5. Cardiotoxicity Assessment Using hERG Assay

Cardiotoxicity potential of BNS808 and BNS822 on the hERG potassium channels was evaluated using the automated patch clamp method (SyncroPatch 384PE). The IC50 values on whole cell hERG currents were summarized in Table 7.

TABLE 7
IC50 values in the hERG assay for BNS808 and BNS822

| Compound ID | IC₅₀(uM) | Hill slope | N |
|---|---|---|---|
| Amitriptyline positive control | 2.58 | 1.05 | 2 |
| BNS808 | 5.39 | 0.89 | 2 |
| BNS822 | >30.00 | — | 2 |

The acceptance criteria for the hERG assay are IC50>100* Ki, meaning that the potential for cardiotoxicity (QT prolongation) was low for both compounds (CB₁ receptor Ki values for BNS808 and BNS822 are 0.6 nM and 1.4 nM, respectively).

6. Hepatotoxicity Assessment Using HepG2 Assay

The liver plays a central role in transforming and clearing chemicals, and therefore is susceptible to toxicity of these agents. BNS808, BNS822 and other selected compounds were tested in the HepG2 assay using human primary hepatocytes to evaluate cytotoxicity. The IC50 data are summarized in Table 8 below.

TABLE 8
IC50 values in the HepG2 assay for selected BNS compounds
Cell Viability Assay in HepG2 Cells at 72 h

| | Absolute | Relative | | | |
|---|---|---|---|---|---|
| | IC₅₀ | IC₅₀ | | | |
| Compound ID | (μM) | (μM) | Top(%) | Bottom(%) | Slope |
| BNS807 | >100 | 17.95 | 21.39 | −12.17 | 1.52 |
| BNS815 | 55.88 | 46.69 | 80.41 | −9.25 | 3.71 |
| BNS825 | >100 | >100 | 6.27 | −11.62 | −0.21 |
| BNS808 | 26.20 | 16.84 | 62.98 | −8.11 | 3.39 |
| BNS822(RD- | >100 | >100 | 11.62 | −7.56 | −0.04 |
| 022-126) | | | | | |
| BNS822(RD- | >100 | >100 | 12.03 | −8.84 | 0.04 |
| 033-002) | | | | | |
| Staurosporine | 0.08 | 0.06 | 71.33 | 1.13 | 2.53 |

The compounds of BNS815 and BNS808 showed cytotoxicity with IC50 at 55.88 μM and 26.20 μM, respectively, and BNS807, BNS825, BNS822(RD-022-126) and BNS822(RD-033-002) showed IC50 over 100 μM compared to the Staurosporine reference with IC50 at 0.08 UM. The acceptance criteria for the HepG2 assay are IC50/Ki>50, meaning the potential for cytotoxicity was low for all tested compounds (CB₁ receptor Ki values for BNS808 and BNS822 are 0.6 nM and 1.4 nM, respectively).

7. Evaluation of CYP Inhibition in Human Liver Microsomes

To evaluate the in vitro CYP inhibition of BNS808 and BNS822, the compounds were tested in human liver microsomes that are rich in drug metabolizing enzymes, including CYPs 1A2, 2C9, 2C19, 2D6, 3A4. The IC50 data in the CYP inhibition model comparing the tested compounds to positive controls are summarized in Tables 9a and 9b below.

TABLE 9a
IC50 values in the HepG2 assay for selected BNS compounds

| ID# | IC50 (μM) | | | | |
|---|---|---|---|---|---|
| BNS808 | >50 | 8.33 | 18.0 | >50 | 2.99 |
| BNS822 | >50 | 8.52 | >50 | >50 | >50 |

TABLE 9b
Inhibition rate (%) in the HepG2 assay for positive controls

| | | | % Inhibition | |
|---|---|---|---|---|
| CYP | | | acceptance | |
| isozyme | Standard inhibitor | % Inhibition | range | Pass/No Pass |
| 1A2 | a-Naphthoflavone | 90.0 | 82.1%-94.9% | Pass |
| 2C9 | Sulfaphenazole | 81.5 | 79.0%-89.9% | Pass |
| 2C19 | (+)-N-3-benzyl- | 84.1 | 77.5%-91.3% | Pass |
| | nirvanol | | | |
| 2D6 | Quinidine | 96.2 | 93.9%-97.0% | Pass |
| 3A4 | Ketoconazole | 98.4 | 97.6%-99.9% | Pass |

The results show that BNS808 is a moderate inhibitor of CYP3A4(M) and weak-moderate inhibitor of CYP2C9 and CYP2C19, and BNS822 is a weak-moderate inhibitor of CYP2C₉.

8. Evaluation of CYP Inhibition in Human Liver Microsomes

The peripherality of selected BB8 conjugates (BNS) was evaluated in vivo measuring their respective brain, plasma, and liver levels 1 hr following 10 mg/kg oral administration. Consistent with the in vitro permeability results, BNS803 (having a high permeablity and not a P-gp substrate) showed a relatively high penetrance to the brain with Brain/Plasma ratio of 1.6. BNS808 (having a low permeability and a P-gp substrate) showed lower brain penetrance with Brain/Plasma ratio of 1.07. BNS822 (having the lowest permeability and a P-gp substrate) showed the lowest brain penetrance with Brain/Plasma ratio of <0.02. Additional parameters of BNS822 oral absorption and pharmacokinetics (PK) were evaluated, demonstrating a moderate absolute oral bioavailability of 8.62 to 10.59 and T_{1/2} of 5.2 hr The results are summarized in Table 10.

TABLE 10
Pharmacokinetic (PK) parameters of BNS compounds

| | Brain/ | Liver/ | T_{1/2} | Tₘₐₓ | Cₘₐₓ | Oral bioavail | Brain protein |
|---|---|---|---|---|---|---|---|
| ID# | Plasma ratio^{a} | Plasma ratio | (h)^{b} | (h) | (ng/mL)^{b} | (%)^{b, c} | binding Fu(%) |
| BNS803 | 1.6 | 8.4 | ND | ND | ND | ND | ND |
| BNS808 | 1.07 | 13.0 | ND | ND | ND | ND | 0 |
| BNS822 | <0.02 | 1.67 | 5.2 | 2 | 283-811 | 8.62-10.59 | 0 |

^{a}Plasma, brain and liver concentrations were determined 1 h post 10 mg/kg oral administration in C57BL/6J male mice.
^{b}PK parameters of BNS822 were determined following oral administration of 10 and 20 mg/kg, in C57BL/6J male mice.
^{c}Absolute oral bioavailability was calculated in comparison to IV administration of 1 mg/kg, in C57BL/6J male mice. Fu = unbound fraction. ND = not determined.

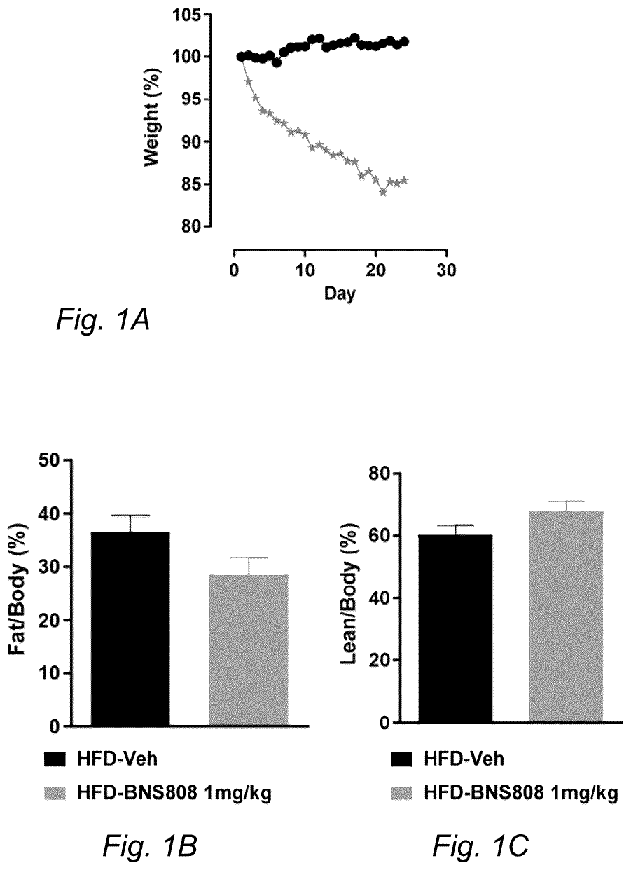

BNS808 showed a very high liver accumulation, with Liver/Plasma ratio of 13.0 (Table 8), suggesting that it may have benefits in ameliorating liver abnormalities associated with obesity. Thus, the efficacy of BNS808 was further evaluated in Diet-Induced-Obesity (DIO) mouse model; while reducing the BNS808 dose to 1 mg/kg in order to avoid high brain concentration. In a mouse obesity model (male C57BL/6J mice fed High-Fat-Diet (HFD) for 25 weeks), mice were started on daily PO administration of BNS808 (1 mg/kg) or vehicle for 3 weeks. The results showed that BNS808 reduced the body weight of mice on HFD, with a significant ˜15% reduction of the body weight after 24 days (FIG. 1A) and a corresponding reduction of adiposity (fat mass) and increase of lean mass (FIGS. 1B and 1C).

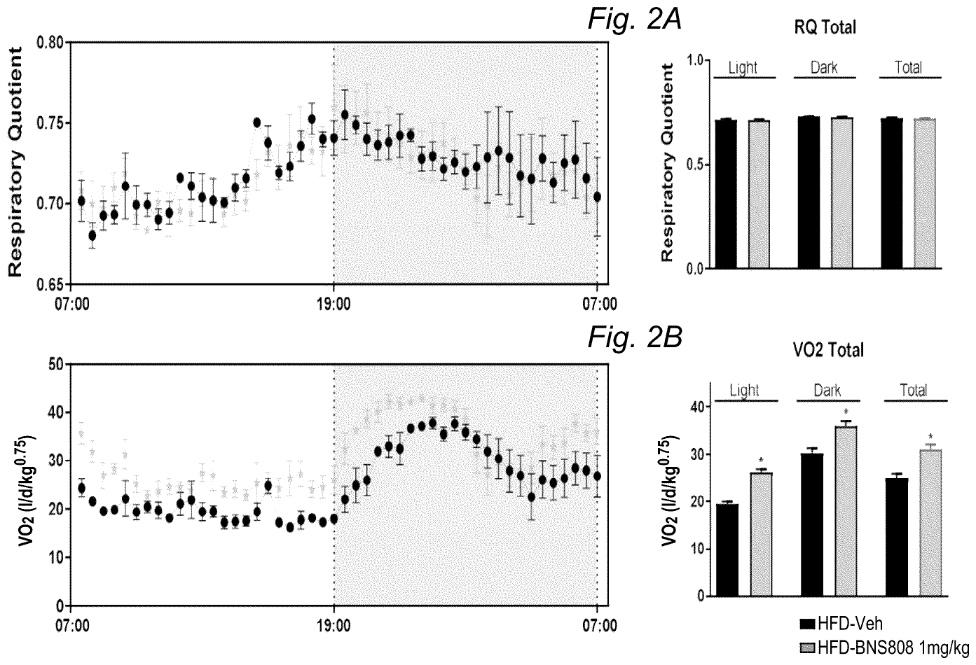

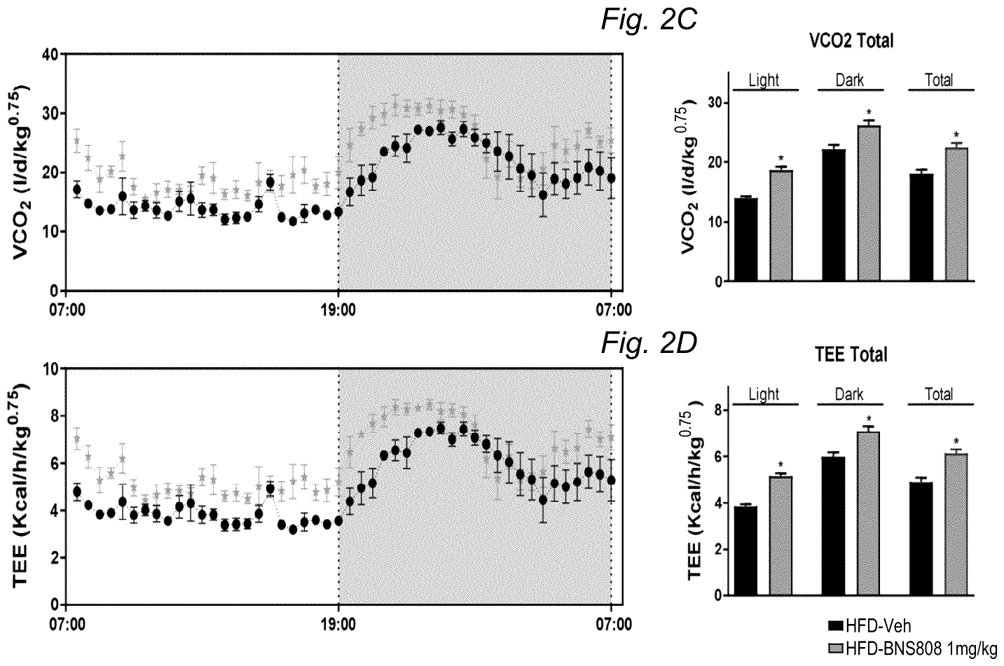

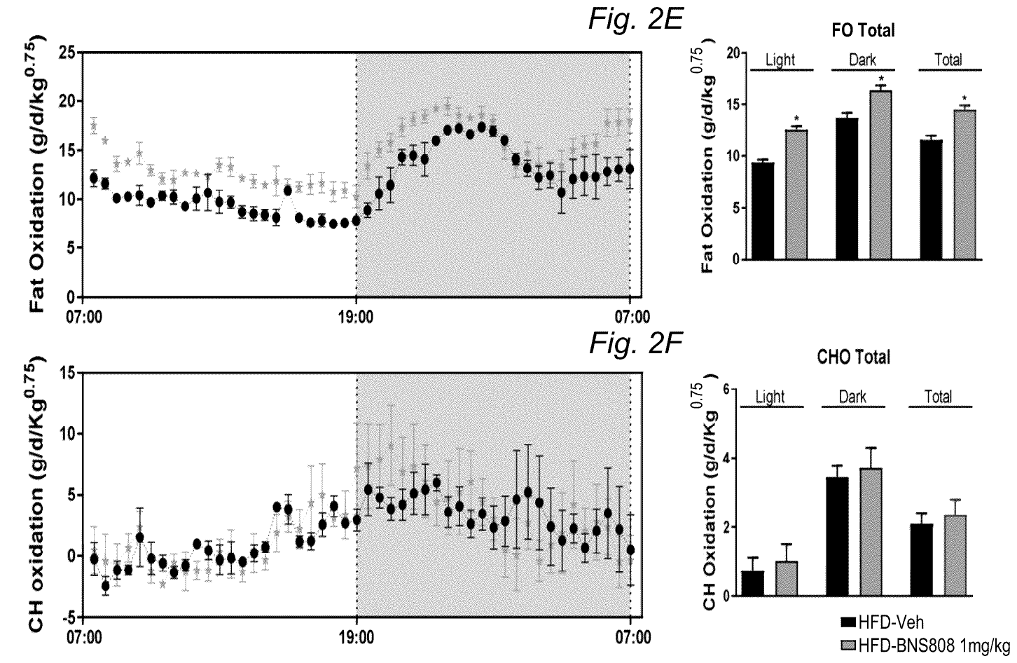

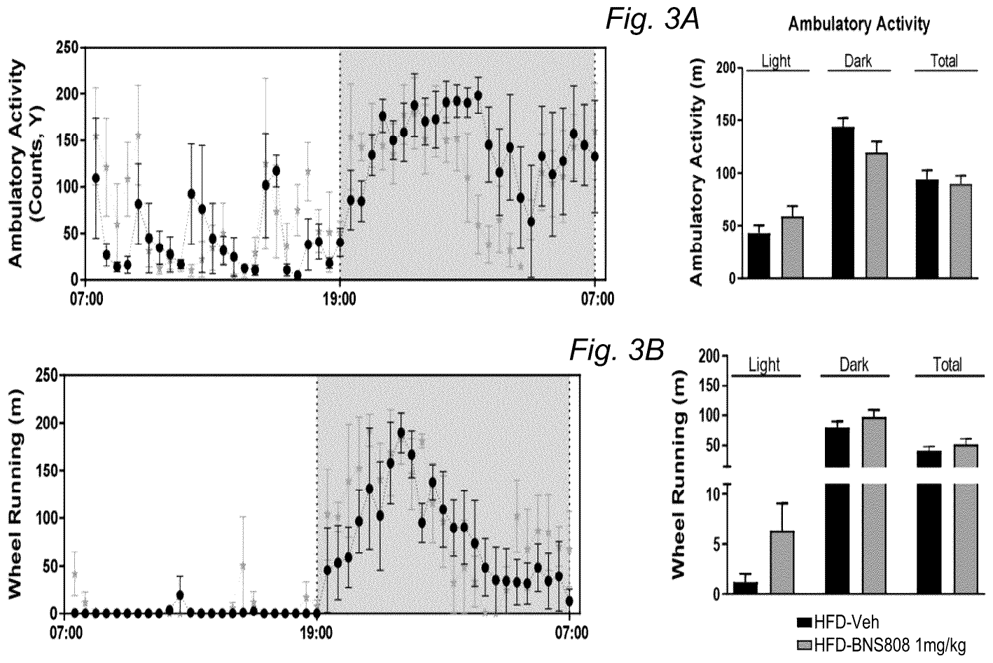

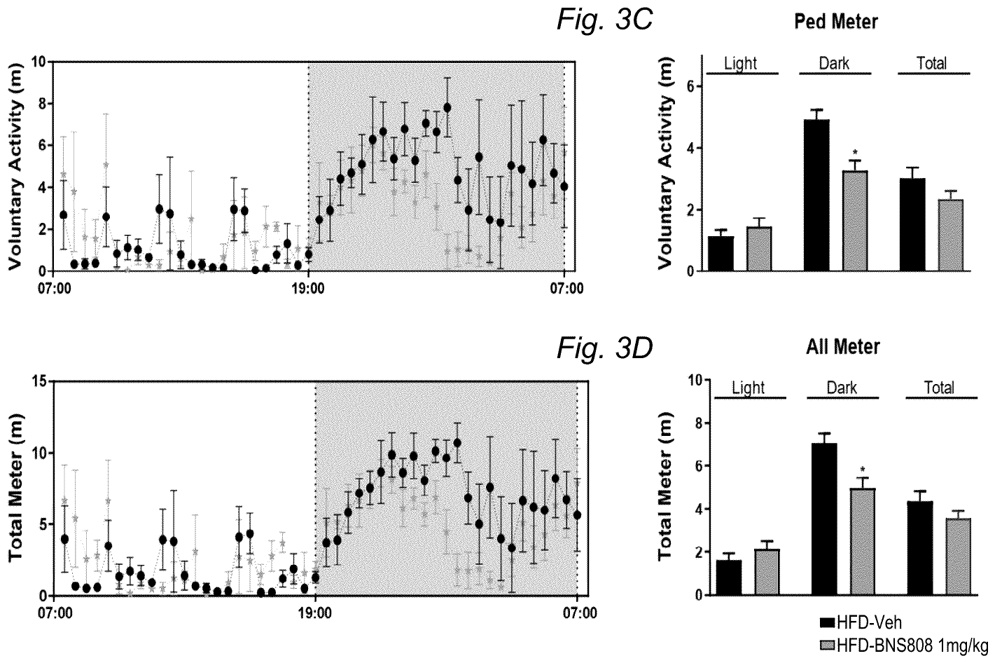

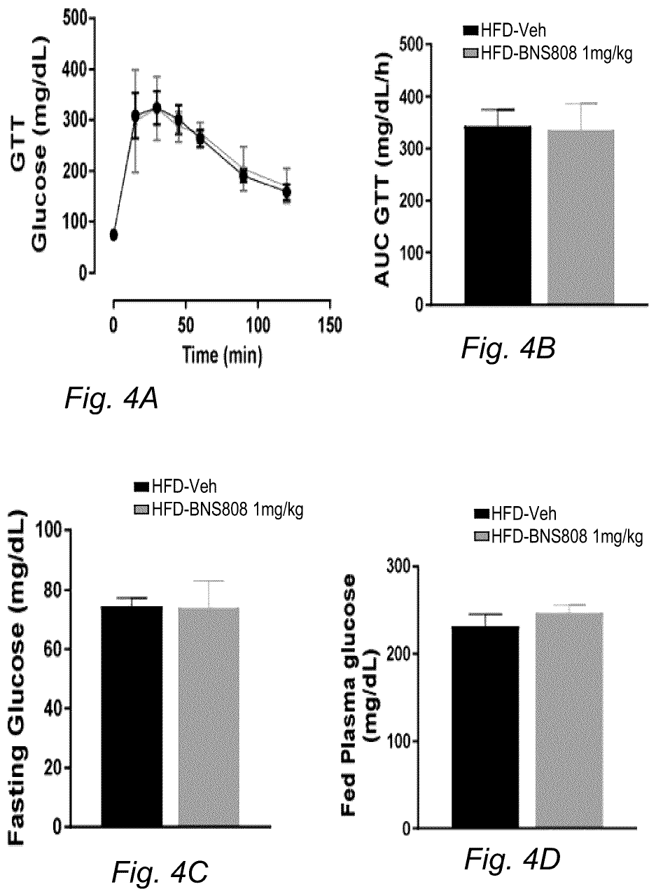

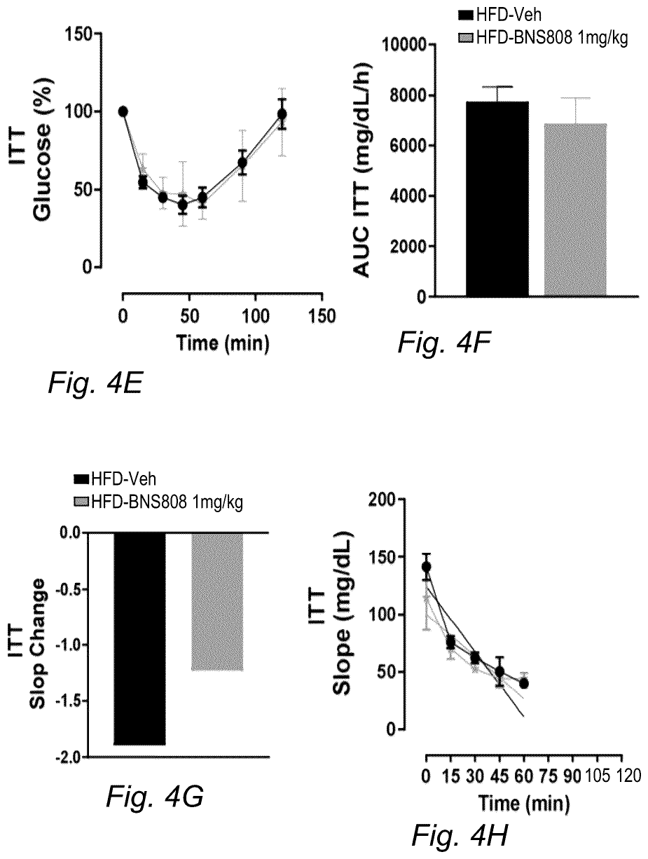

Further, using an indirect calorimetric assessment of the DIO mice, it was shown that BNS808 significantly increases the metabolic profile in upregulating VO₂, VCO₂, total energy expenditure (TEE), and fat oxidation (FIGS. 2A-2F). Importantly, continued PO administration of BNS808 did not affect ambulatory activity of DIO mice (FIGS. 3A-3D). In addition, continued PO administration of BNS808 did not impact on HFD-induced hyperglycemia and glucose intolerance, showing the same glucose levels as the control group (FIGS. 4A-4D). Certain trend was observed toward an increase in insulin sensitivity (FIGS. 4E-4H).

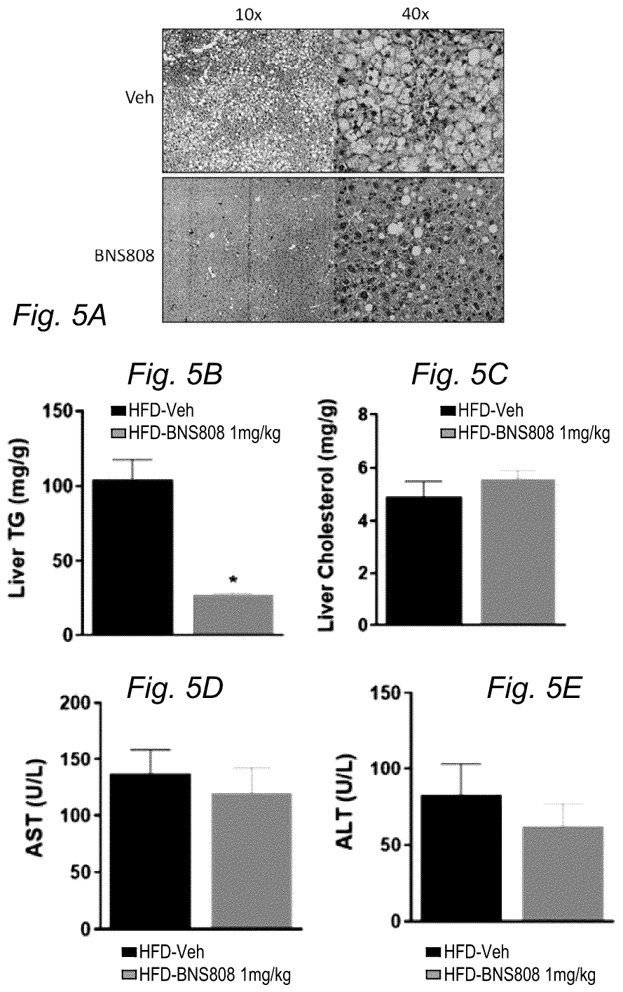

Still further, BNS808 was able to ameliorate HFD-induced hepatic steatosis, reflected in the reduction of fat vacuoles in the liver (FIG. 5A) and the reduction of liver triglycerides (TG) (FIG. 5B)). Certain trend was observed toward an improvement of liver injury, reflected reduced liver enzymes, ALT and AST (FIGS. 5D-5E).

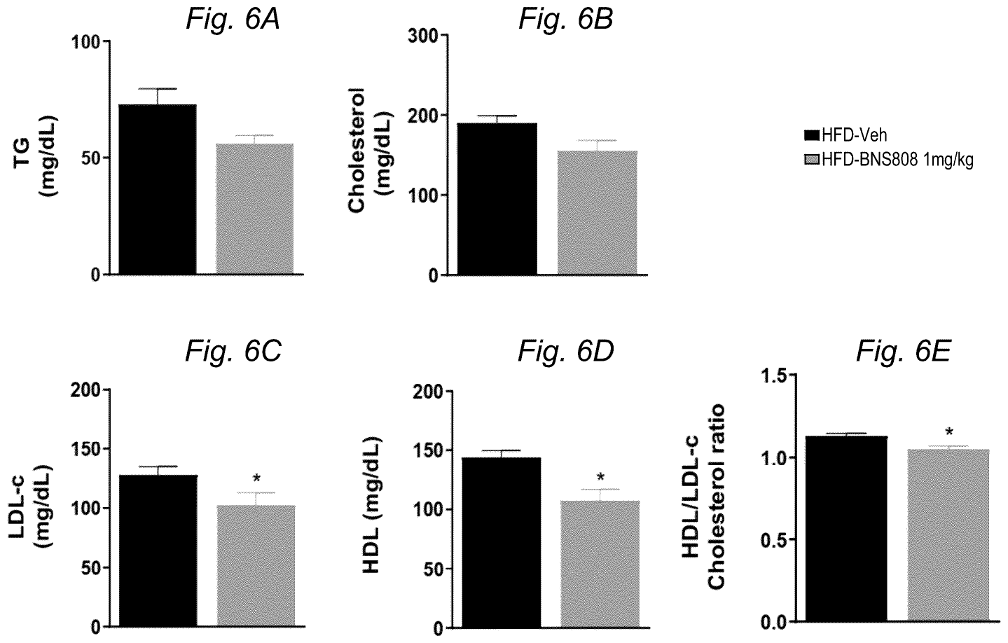

The effect of BNS808 on HFD-induced dyslipidemia was evaluated measuring TG, cholesterol, LDL and HDL serum levels. BNS808 was related to partial improvement of dyslipidemia, by a significant reduction in LDL levels (FIG. 6C) and corresponding trends in TG and cholesterol serum levels (FIGS. 6A-6B). Certain decrease in HDL levels was also observed (FIG. 6D).

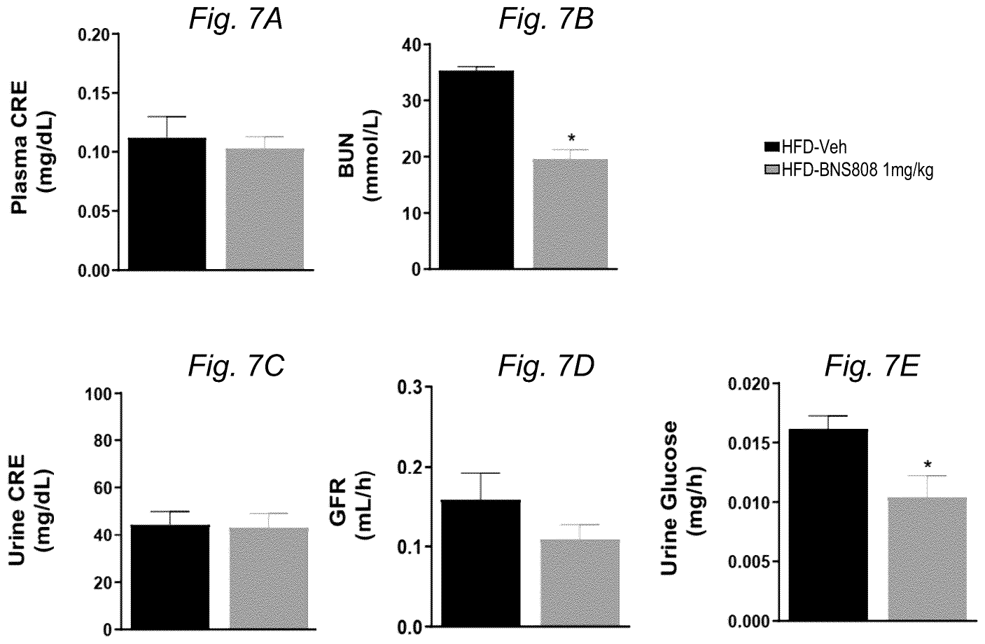

The effect of BNS808 on kidney function was further evaluated, finding no effects on plasma and urine levels of creatinine (FIGS. 7A and 7C) but significant decreases in blood urea nitrogen (BUN) (FIG. 7B) and urine glucose (FIG. 7E), and certain trend toward improvement of HFD-induced kidney hyperfiltration (FIG. 7D).

PK was measured following oral administration of BNS822 at 10 and 20 mg/kg in mice, the PK parameters are shown in Table 8. Plasma peak concentrations were achieved after 2 hr, with Cmax values in the ranges between 283 to 811 ng/ml for the 10 and 20 mg/kg dosing, respectively. BNS822 oral dose of 10 mg/kg) corresponded to brain tissue levels of 1.34 ng/g 1 hr administration, suggesting minimal brain penetration.

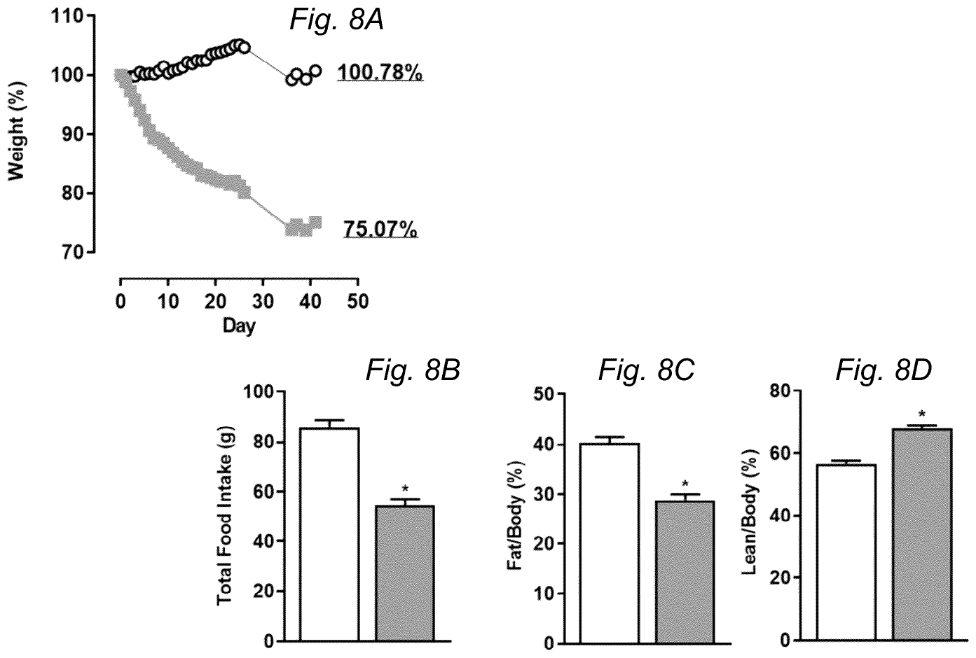

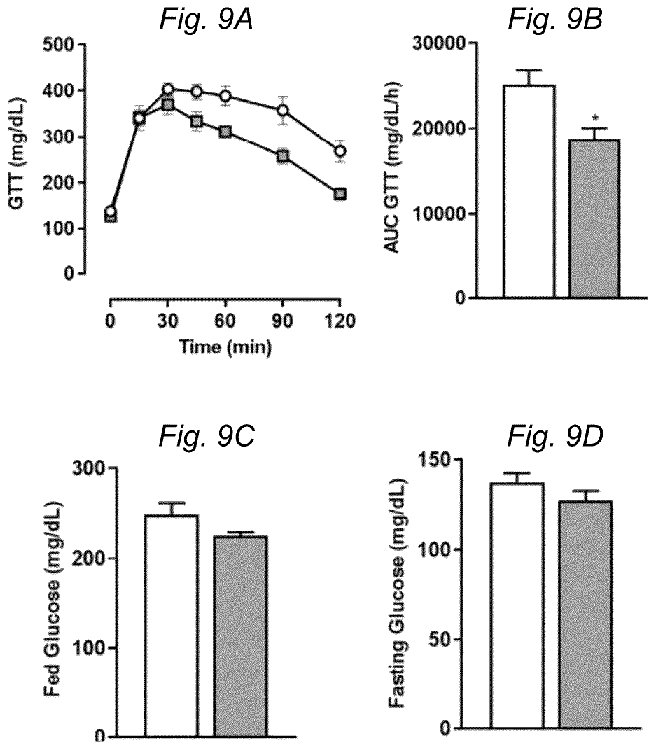

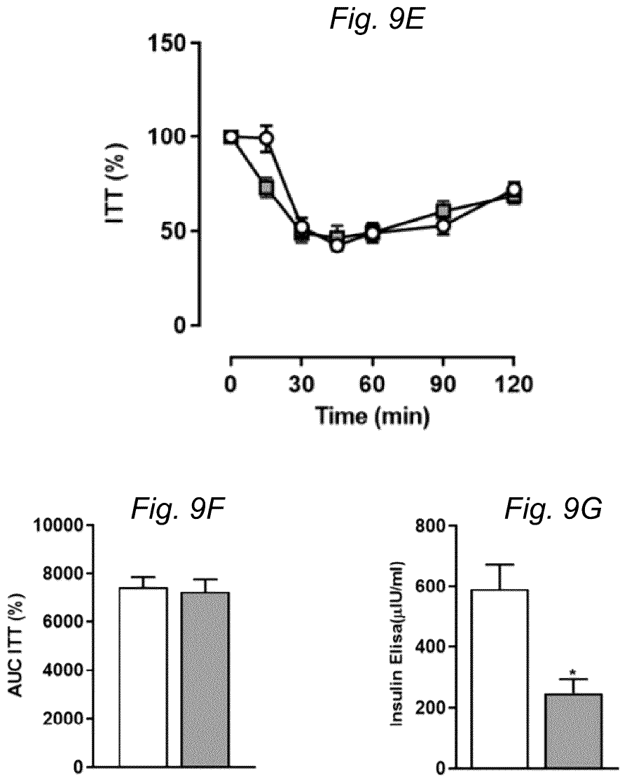

To increase drug exposure in further studies in the DIO model, the chosen dosing level was 20 mg/kg. Under these conditions, continued administration of BNS822, significantly reduced the body weight of DIO mice (FIG. 8A), resulting in 20% weight loss and a significant decrease in food intake (FIG. 8A), and corresponding, significant changes in fat and lean mass (FIGS. 8C-8D). In addition, continued administration of BNS822 improved glucose tolerance in DIO mice evident by AUC in glucose tolerance test (GTT) (FIGS. 9A-9B), with no change in glucose levels (both in fed and fasting conditions) and no improvement in insulin sensitivity (FIGS. 9C-9F). However, circulating fed insulin levels in BNS822 treated mice were significantly lower than control (FIG. 9G), supporting the notion of improvement in the glucose intolerance.

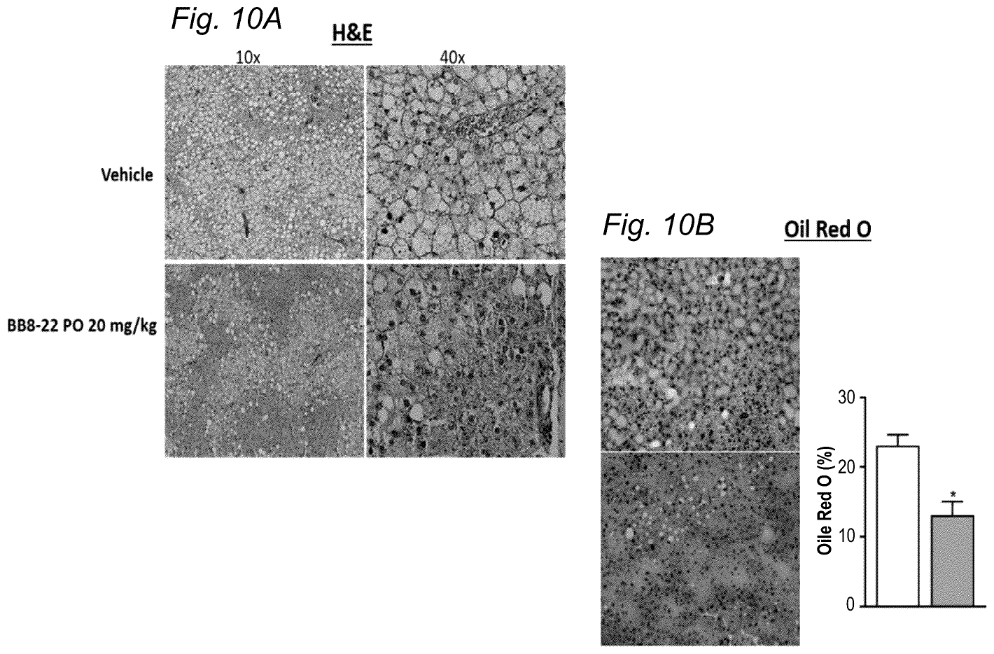

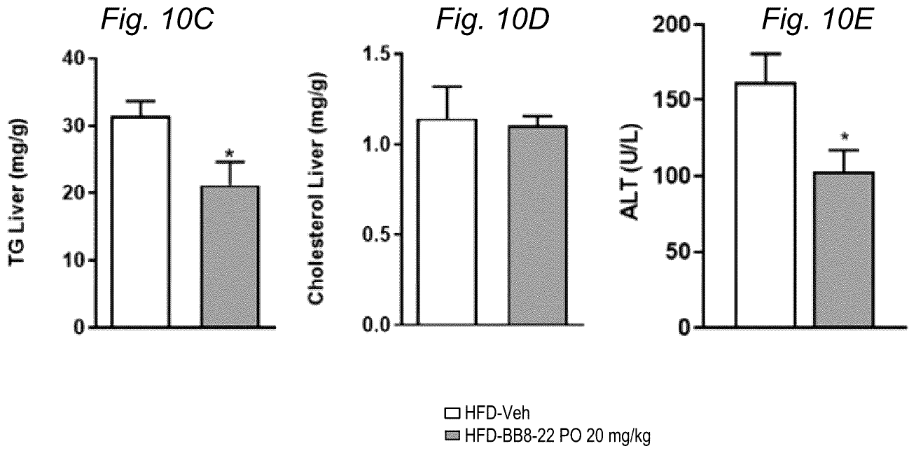

Further, BNS822 treatment was related to improved parameters of HFD-induced hepatic steatosis and liver injury, reflected in a reduction of fat vacuoles deposition in the liver (FIG. 10A), recovery of the hepatic tissue by oil red oil staining (FIG. 10B), and a significant reduction in liver TG content and ALT levels (FIGS. 10C and 10E) but not in the liver cholesterol (FIG. 10D).

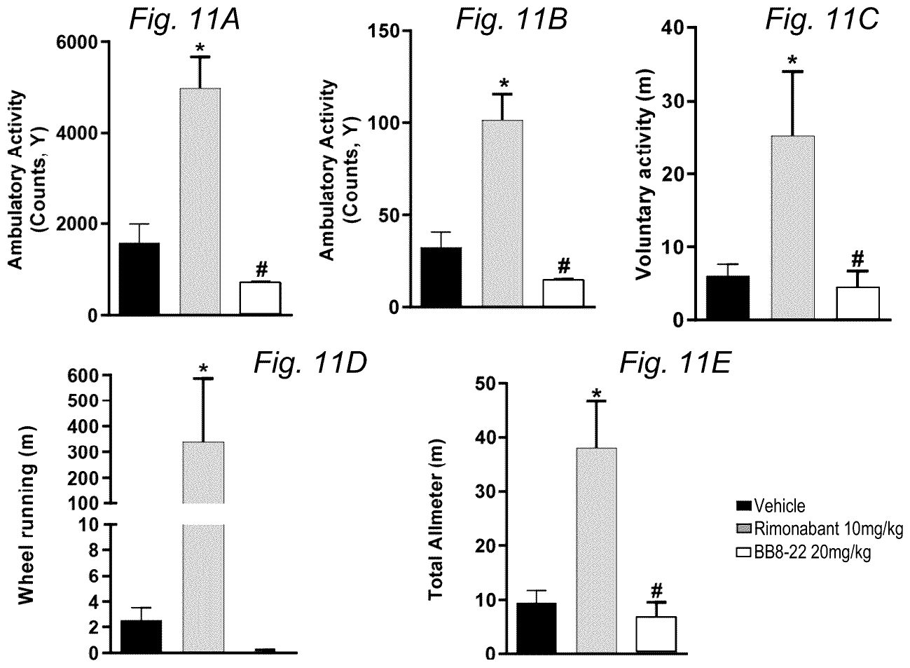

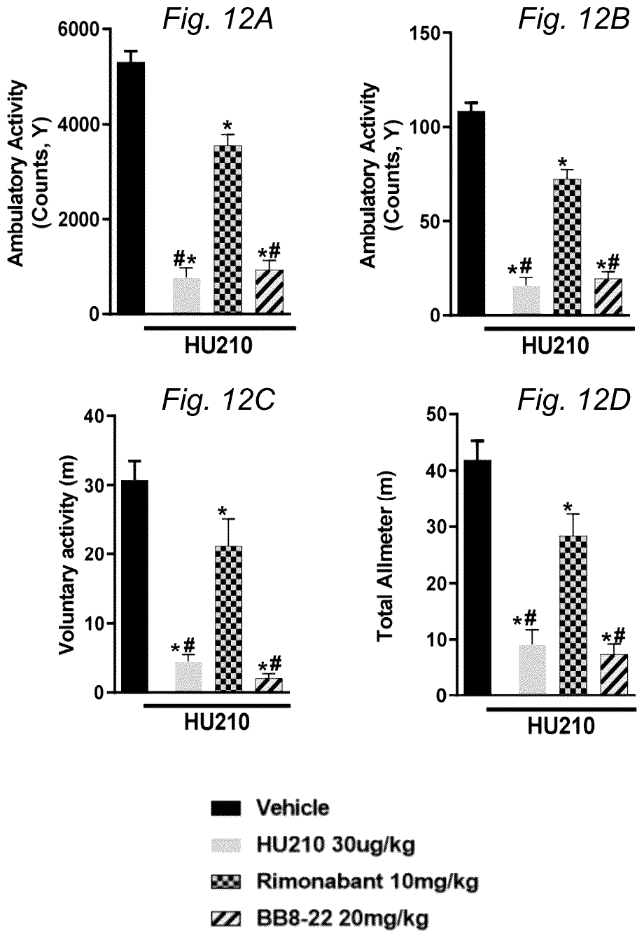

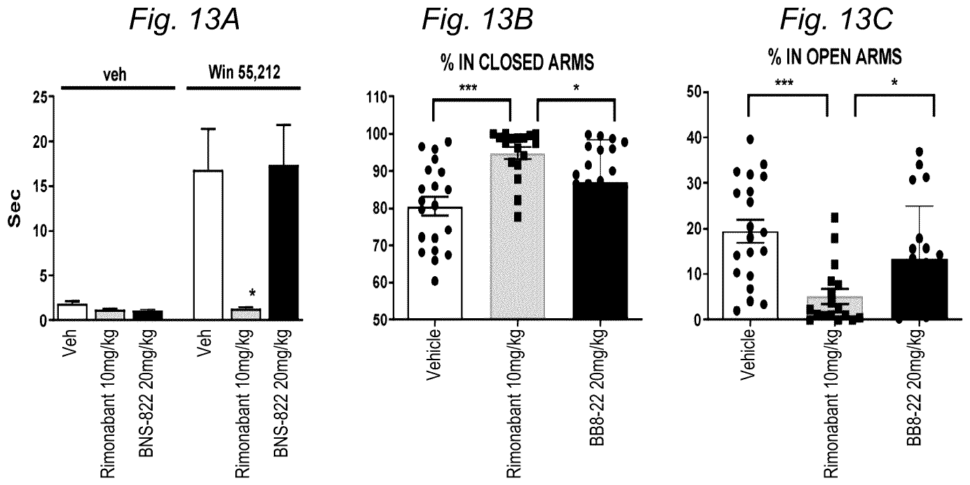

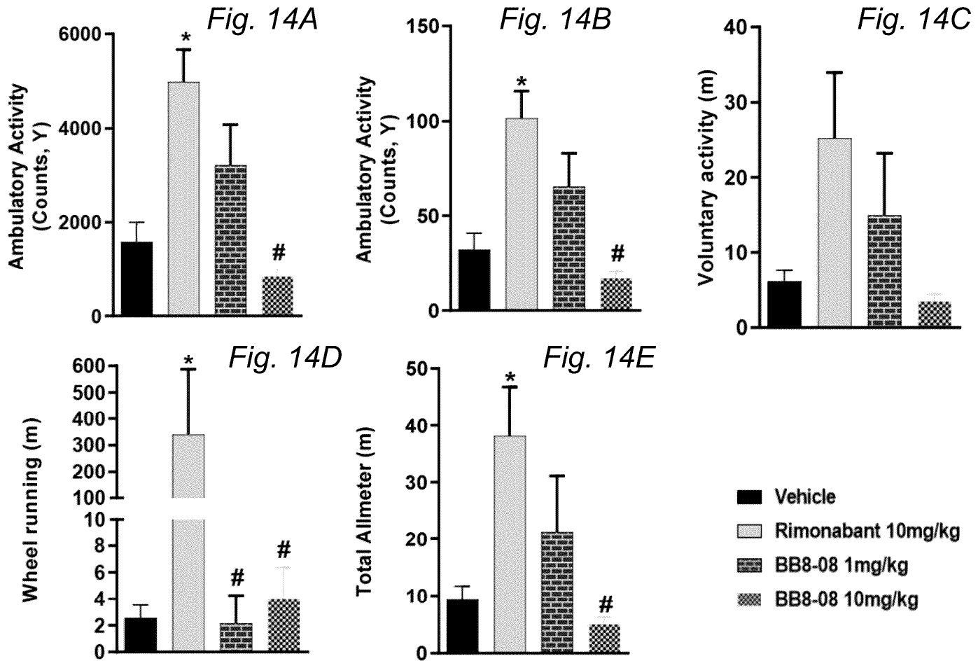

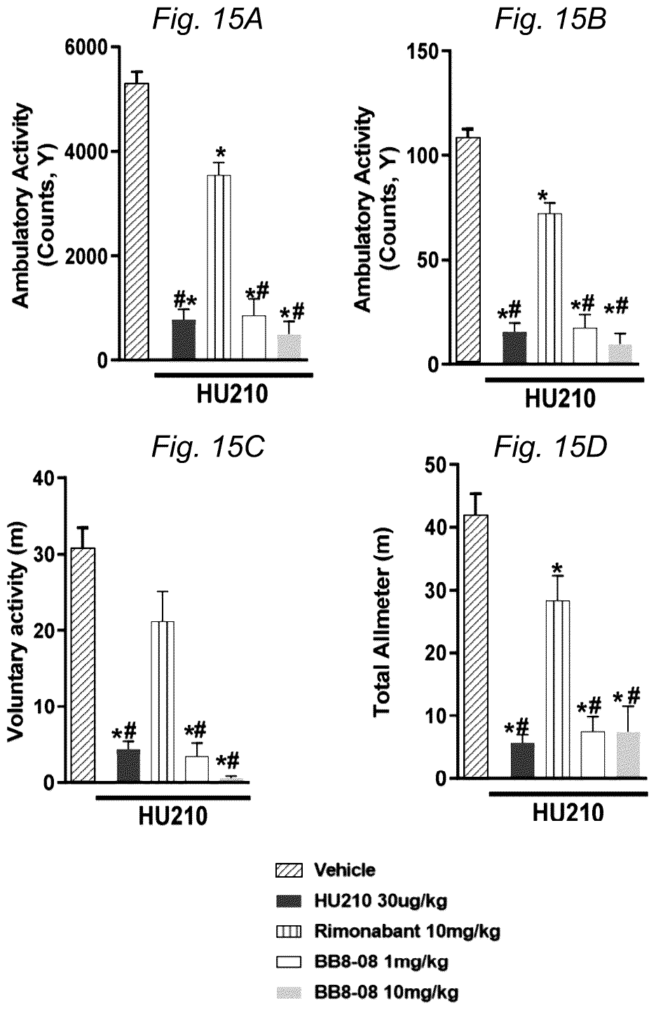

The ability of BNS822 and BNS808 to induce CNS-mediated hyperactivity was further evaluated in in a mouse model, using rimonabant (a brain penetrant CB₁ receptor blocker) as a positive control. Mice (wild-type male C57Bl/6J) received a single dose of rimonabant (10 mg/kg IP), BNS822 (20 mg/kg PO) BNS 808 (1, 10 mg/kg PO) or vehicle; ambulatory activity was measured by the Promethion Metabolic System (Sable Instruments, Inc). In the same experimental scheme, BNS822, BNS808 and rimonabant were further studied regarding the potential to inhibit the hypomotility-induced by a CB₁ receptor agonist, HU210, after a single dose of rimonabant (10 mg/kg IP), BNS822 (20 mg/kg PO) BNS808 (1, 10 mg/kg PO) or vehicle and a single dose of HU210 (30 μg/kg IP) 30 min after. In this framework, unlike rimonabant, BNS822 and BNS808 showed no locomotor effects, evident by apparent lack of effects on locomotor activity (FIGS. 11 and 14) and HU210-induced hypomotility (FIGS. 12 and 15). Moreover, when further tested in behavioral paradigms using WIN-55,212 (a potent cannabinoid receptor agonist) (3 mg/kg IP), unlike rimonabant, BNS822 did not block the WIN-55,212 mediated cataleptic behavior (FIG. 13A) nor did it induce a robust anxiogenic response in the elevated plus maze (EPM) paradigm (FIGS. 13B and C).

1.-40. (canceled)

41. A compound of formula (I): 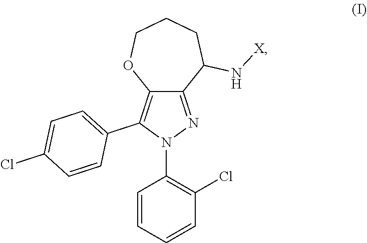
wherein
X is a group selected from —SO₂—R₁, —(C═O)—R₁, —(C═O)-Cyc-(C═O)—R₁, —(C═O)-Cyc-R₁, —(C═O)-Cyc-SO₂—R₁, and —(C═O)-Cyc,
each R₁ is selected independently from —C₁-C₅alkyl, —C₆-C₁₀aryl, —C₃-C₆ heteroaryl, —C₅-C₁₀carbocyclic, —C₃-C₆heterocarbocyclyl and NRR′R″, wherein
each of R, R′ and R″, independently, is selected from substituted or unsubstituted —C₁-C₅ alkyl, —C₆-C₁₀aryl, —C₃-C₆heteroaryl, —C₅-C₁₀carbocyclic, —C₃-C₆heterocarbocyclyl, —C₆-C₁₀arylene and —C₃-C₆ heteroarylene.

42. The compound according to claim 41, wherein each group R₁ is substituted by one or more functional groups.

43. The compound according to claim 42, wherein the substituting functional group is selected from halides (Cl, Br, I and/or F), hydroxyl groups, amines, nitro groups, nitrile groups, sulfoxide groups, sulfonyl groups, alkyl groups, alkenyl groups, alkynyl groups, trifluorinated groups, aldehyde groups, ester groups, ketone groups and amide groups.

44. The compound according to claim 41, wherein X is any one of

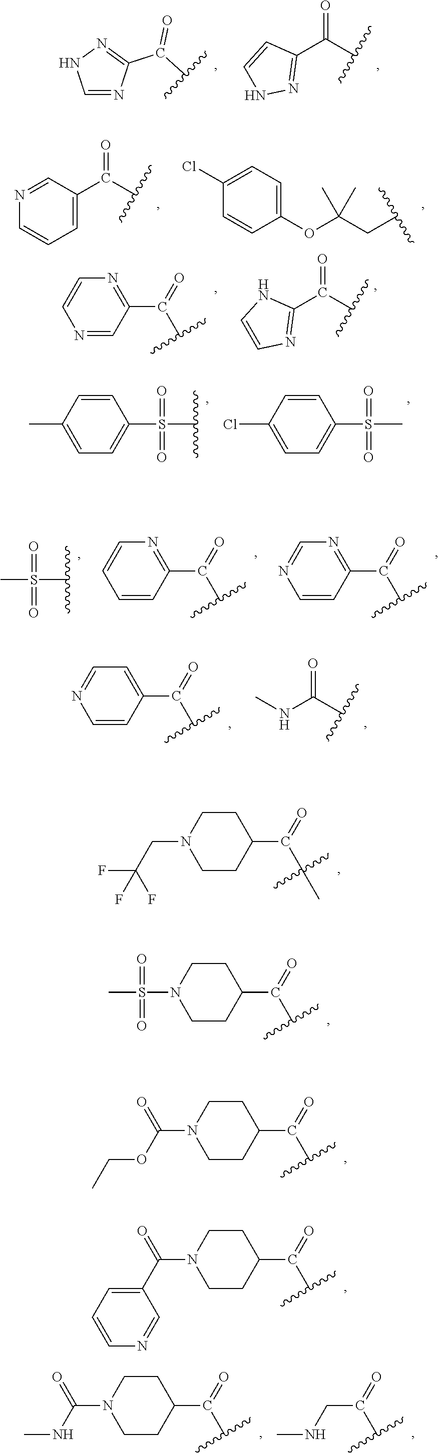

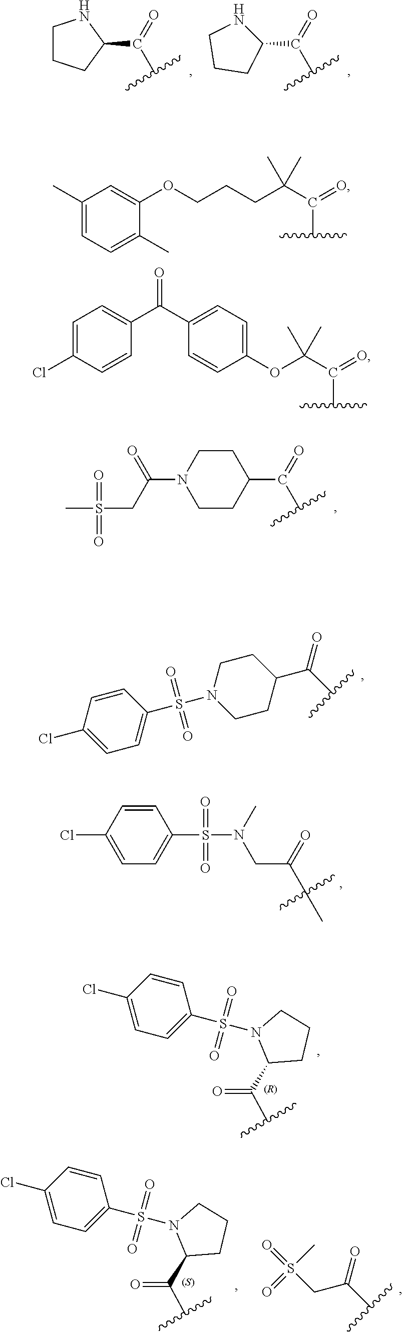

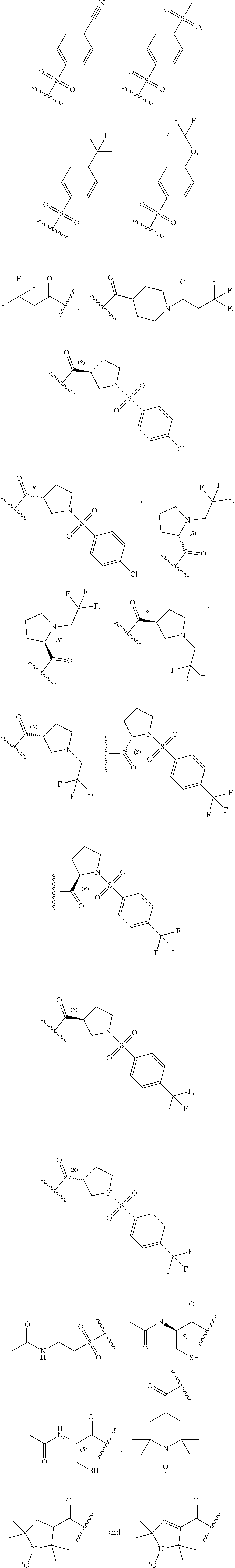

45. The compound according to claim 41, being a compound herein designated BNS801 through BNS828.

46. The compound according to claim 48, being a compound designated BNS808 and BNS822.

47. A composition comprising a compound according to claim 41.

48. A nanocarrier comprising at least one compound according to claim 41.

49. A method of preventing, alleviating or treating a metabolic disease, disorder or condition in a subject in need thereof, the method comprising administering to the subject a composition according to claim 47.

50. The method according to claim 49, wherein the metabolic disease, disorder or condition comprises at least one of obesity, insulin resistance, diabetes, coronary heart disease, liver cirrhosis, fatty liver disease, chronic kidney disease and/or cancer.

51. The method according to claim 49, wherein preventing, alleviating or treating a metabolic disease, disorder or condition comprises at least one of a reduction in a body weight, a reduction in a body fat, a reduction in blood pressure (hypertension), a reduction in the serum levels of LDL (low-density) cholesterol, an increase in the serum levels of HDL (high-density) cholesterol, an increase in the serum HDL/LDL cholesterol ratio, an increase in the serum triglycerides.

52. The method according to claim 49, wherein the administering of the composition, the pharmaceutical composition or the formulation to the subject is an oral administering.

53. The method according to claim 49, wherein the administering of the composition, the pharmaceutical composition or the formulation to the subject is IV (intravenous) or IM (intramuscular) administering.

54. The method according to claim 49 further comprising administering of at least one additional therapeutic agent for preventing, alleviating or treating the metabolic disease, disorder, condition in the same subject.

55. The method according to claim 49, wherein the subject is a human or a mammal.

56. A method of preventing, alleviating or treating a weight gain in a subject in need thereof, the method comprising administering to the subject a composition according to claim 47.

57. The method according to claim 56, wherein said administering of the composition, pharmaceutical composition or the formulation to the subject is oral administering.

58. The composition according to claim 47, wherein the compound is designated BNS808 and BNS822.

59. The method according to claim 49, wherein the compound is designated BNS808 and BNS822.
